(19) United States
(12) Patent Application Publication
(10) Pub. No.: US 2010/0061964 A1
(43) Pub. Date: Mar. 11, 2010

(54) METHOD FOR PREPARING PANCREATIC BETA CELLS STARTING FROM BETA CELL PROGENITORS AND BETA CELLS THEREBY OBTAINED

(76) Inventors: Harry Heimberg, Flobecq (BE); Xiaobo Xu, Jette (BE)

Correspondence Address: Craig J Arnold;Amster Rithstein & Ebenstein, 90 Park Avenue, New York, NY, 10016, US

(21) Appl. No.: 12/448,701

(22) PCT Filed: Jan. 4, 2008
(86) PCT No.: PCT/EP2008/050069

§ 371 Date: Jul. 1, 2009

(30) Foreign Application Priority Data
Jan. 5, 2007 (EP) PCT/EP2007/000065

(51) Int. Cl. A61K 35/12; C12N 5/00; A01N 1/00; C12Q 1/02; A61K 38/16; C07K 14/00; C07K 1/14; A61P 3/10

(52) U.S. Cl. 424/93.7; 435/377; 435/325; 435/1.1; 435/29; 514/12; 530/350; 530/412

(57) ABSTRACT

The present invention relates to a method for preparing pancreatic islet cells, and preferably pancreatic beta cells starting from progenitor cells obtained from the pancreas of an adult mammal. The invention also provides a method for isolating progenitor cells, and preferably beta cell progenitors, from the pancreas of an adult mammal. Other aspects of the invention comprise the pancreatic islet (beta) cells and the progenitor cells that can be obtained with the present invention and their use, e.g. in diagnostic, therapeutic or research applications.

### FIELD OF THE INVENTION

The present invention relates to the medical field. More in particular, the invention is directed to a method for preparing pancreatic beta cells starting from beta cell progenitors and the beta cells thereby obtained. The present invention also provides a method for isolating beta cell progenitors and to progenitors thereby obtained. In addition, the invention relates to methods for the differentiation and proliferation of such progenitors to beta cells and applications of so differentiated and proliferated beta cells.

### BACKGROUND

Diabetes mellitus is a disease of the glucose regulatory system characterized by hyperglycemia (high glucose blood sugar), among other signs. There are two main types of diabetes. Type 1 diabetes (also called juvenile onset or insulin-dependent diabetes) is due to an autoimmune attack on the insulin secreting pancreatic beta-cell. Type 2 diabetes (also called adult onset or non-insulin-dependent diabetes) is characterized by a reduced mass of beta-cells, reduced insulin secretion and resistance to the action of insulin (elevated concentrations of glucose in the blood.

Destruction or dysfunction of beta cells is the cause of diabetes mellitus type 1. In Diabetes mellitus type 2 beta cells decline gradually over time, and insulin resistance plays a role in the disease. Type 2 sometimes progresses to loss of beta cell function as well.

Beta cells are a type of cell in the pancreas in areas called the islets of Langerhans. These islets of Langerhans regroup the endocrine (i.e., hormone-producing) cells of the pancreas The islets are a compact collection of endocrine cells arranged in clusters and cords and are crisscrossed by a dense network of capillaries. Hormones produced in the Islets of Langerhans are secreted directly into the blood flow by (at least) four different types of cells: Beta cells producing Insulin and Amylin (65-80% of the islet cells); Alpha cells releasing Glucagon (15-20%); Delta cells producing Somatostatin (3-10%); PP cells containing polypeptide (1%). Islets can influence each other through paracrine and autocrine communication, and beta-cells are coupled electrically to beta-cells (but not to other cell-types).

Much research is being done in the field of beta-cell physiology and pathology. One major research topic is its effects on diabetes. Many researchers are trying to find ways to use beta-cells to help control or prevent diabetes. A major topic is the replication of adult beta-cells and the application of these to diabetes.

The present application aims to provide another approach using beta cells to help control or prevent diseases associated with reduced islet (beta) cell functioning and/or reduced islet (beta) cell mass, such as e.g. diabetes.

### SUMMARY OF THE INVENTION

The present invention is directed to a method for preparing pancreatic islet cells, among which are alpha-cells, beta-cells, delta-cells and pp-cells that can be used in the treatment and/or diagnosis of diabetes. In a preferred embodiment, the islet cells are pancreatic beta cells.

More in particular, in a first aspect the invention is directed to a method for preparing pancreatic islet (beta) cells starting from progenitor cells present in adult mammals.

In another aspect the invention is directed to pancreatic islet cells among which are alpha-cells, beta-cells, delta-cells and pp-cells that can be obtained with the present method and their diagnostic and/or therapeutic application, and/or their use in fundamental research. In a preferred embodiment, the islet cells are pancreatic beta cells.

In still another aspect, the invention also provides a method for isolating progenitor cells from adult pancreatic tissue, and to the pancreatic progenitor cells, among which are alpha-cells, beta-cells, delta-cells and pp-cells that can be obtained with such method and to the diagnostic and/or therapeutic application of such progenitor cells and/or their use in fundamental research. In a preferred embodiment, the islet cells are pancreatic beta cells.

In addition, the present invention further provides pharmaceutical compositions and method of treating and/or diagnosing disease associated with reduced pancreatic islet cell functioning and/or reduced pancreatic islet cell mass, such as diabetes, e.g. of type I and/or type II. The pancreatic islet cells are selected from the group of alpha-cells, beta-cells, delta-cells and pp-cells. In a preferred embodiment, the cells are beta-cells.

The present invention is at least in part based on the unexpected finding that progenitors of pancreatic islet cells such as beta-cells can be found in adult pancreas tissue. This finding is rather surprising, especially in view of earlier reports describing that adult pancreatic beta cells are formed by self-duplication rather than differentiation from progenitor (stem) cells, as was for instance reported in Dor and Melton (Nature, 429, 41-46, 2004), who excluded endogenous progenitor (stem) cells as a source of beta cells in adult pancreas and concluded that embryonic stem cells are the “only type of stem cell that is unquestionably capable of differentiation into beta cells” and in Teta et al (Dev. Cell, 12, 817-826, 2007), that explicitly states that growth and regeneration of adult beta cells does not involve specialized progenitor cells.

The present invention is at least in part based on the following novel findings:
The applicants have demonstrated that beta cell progenitors are present in adult mouse pancreas. These cells can be are traced in adult Neurogenin-3 (Ngn3) promoter-reported mice following partial duct ligation. The present progenitors can be isolated and characterized as bona fide stem/progenitor cells and were shown to be able to differentiate in functional beta cells.
The application has further shown that the Ngn3-expressing beta cell progenitors are located among non-endocrine cells lining the pancreatic duct.
The Ngn3 activity is causally related to beta cell hyperplasia as evidenced by the effect of Ngn3-specific known down following partial duct ligation.
The Ngn3-expressing beta cell progenitors allow an increase in beta cell mass through their differentiation rather than by proliferation of pre-existing beta cells.
The Ngn3-expressing cells are not the result of dedifferentiation of differentiated endocrine pancreatic cells
The purified Ngn3 cells from adult pancreas, able to start insulin expression when transplanted in vitro in a pancreatic extract are not the result of cell fusion

The present application provides evidence that endogenous multipotent progenitor cells are present in adult pancreatic tissue and can be activated to give rise to functional insulin-producing beta cells. In this respect, the present findings are useful in the context of developing novel therapeutic strategies in diabetes: hereby efforts to generate beta cells should be balanced between embryonic and adult stem cell research.

The beta cell model system provided in the invention is further used to determine the physiological or in vivo conditions of beta cell regeneration and identifies the extracellular or factors such as chemokines, and growth factors that induce beta cell differentiation of progenitors in an adult pancreas. It is without saying that the identification of these factors will make it possible to design treatments, pharmaceuticals, drugs or medicaments, enabling regeneration of progenitor cells into beta cells in a patient suffering from a decrease beta cell mass such as type 1 diabetes mellitus.

The invention further provides for an experimental set-up that can result in the production of beta cells in vitro, which can be transplanted into diseased subjects in need thereof.

With the insight to better show the characteristics of the invention, some preferred embodiments and examples are described hereafter referring to the enclosed drawings.

### DETAILED DESCRIPTION OF THE DRAWINGS

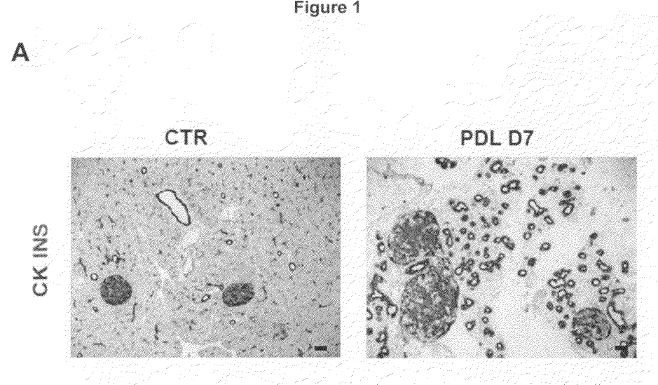

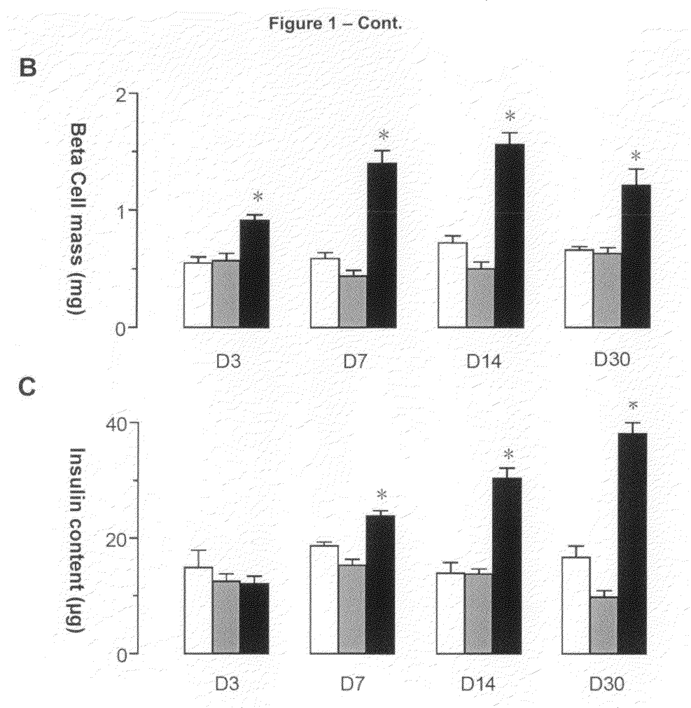

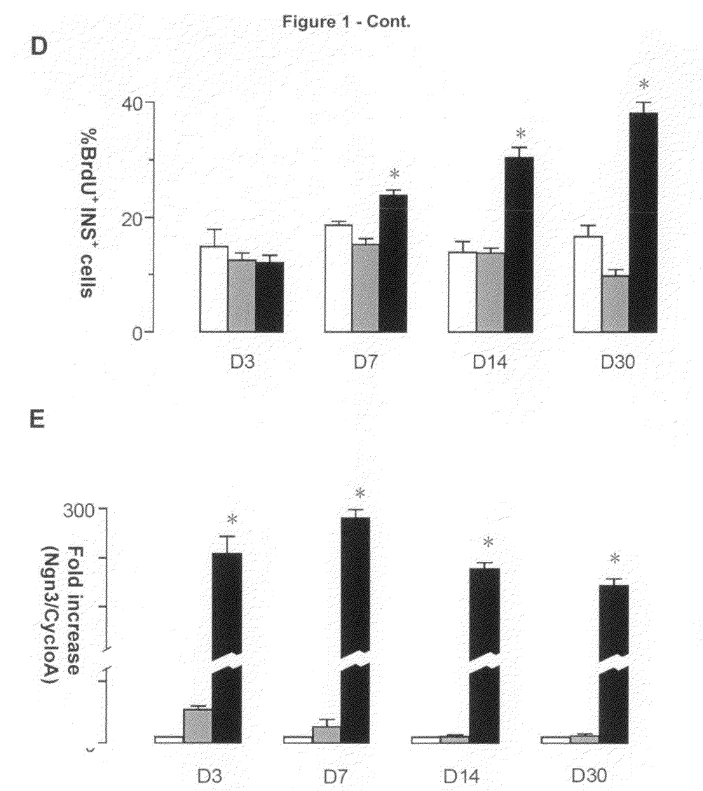

FIG. 1: PDL ACTIVATES NGN3 GENE EXPRESSION AND INCREASES BETA CELL MASS IN ADULT PANCREAS. In 8 weeks old BALB/C mice, the duct that connects pancreatic tail and duodenum was ligated and the ligated tail of PDL pancreas at day 7 (PDL D7) was compared to the tail of sham-treated pancreas (CTR) by immunohistochemistry for cytokeratin+ (CK) ductular complexes and insulin+ cells (A). Magnification bars are 100 μm. Several parameters were measured (see Experimental Procedures) at 3, 7, 14 and 30 days following ligation. PDL increased the insulin+ cell mass (mg) (B) and the insulin content (C) (mg) of the tail part of pancreas more than 2-fold (black bars) as compared to the unligated head of the same pancreas (grey bars) and the tail of a sham-operated pancreas (white bars). (D) At 3, 7, 14 and 30 days after PDL and 1 hour before sacrifice, the nucleotide analogue BrdU (50 mg/kg) was injected intra peritoneum. The number of insulin+ BrdU+ cells on pancreatic tissue sections was 10-fold higher in the ligated tail vs control tail or head of pancreas. A similar relative increase of BrdU+ beta cells was seen when BrdU had been applied 16 hours before sacrifice (4.60±0.51% in ligated tail of pancreas at day 7 following PDL vs. 0.66±0.15% in unligated tail). (E) A more than 50-fold increase of Ngn3 transcripts was observed in ligated vs unligated part of pancreas by real time RT-PCR using a mouse Ngn3-specific TaqMan probe. All results shown are representative of 3 or more independent experiments. *: p<0.001 ligated vs unligated pancreas tail.

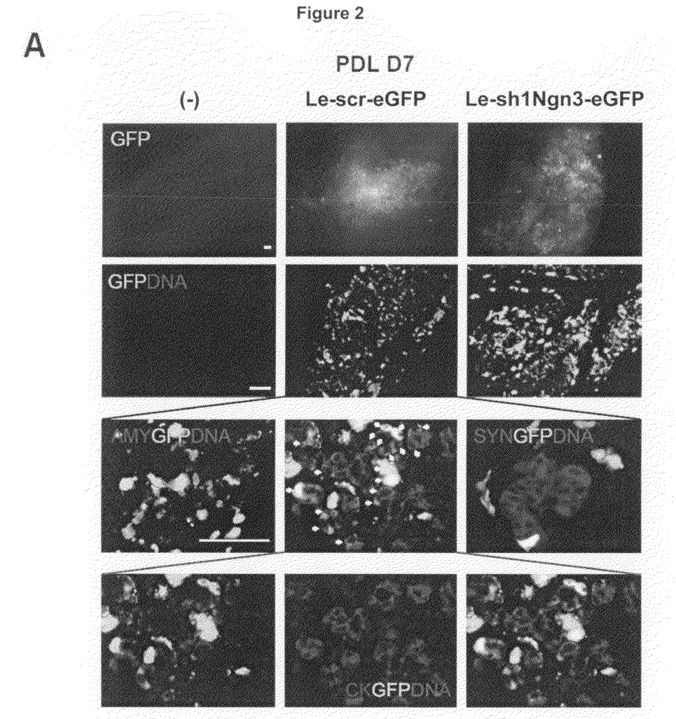

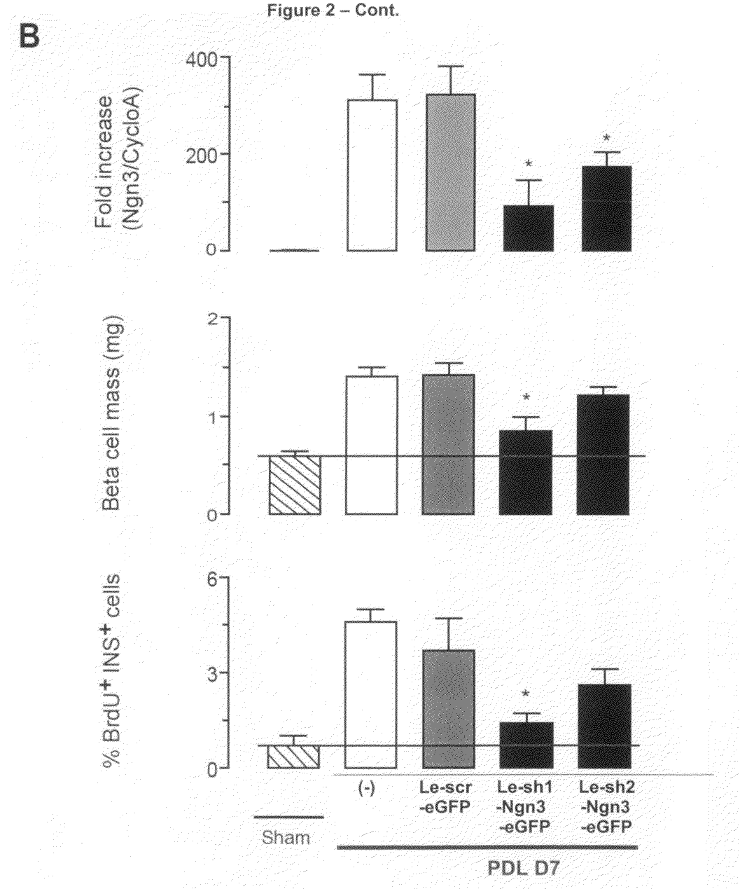

FIG. 2: KNOCK-DOWN OF NGN3 IMPAIRS PDL-INDUCED BETA CELL GENERATION. (A) The pancreatic duct of adult BALB/C mice was injected with recombinant lentiviruses encoding reporter eGFP and 2 different short hairpin RNAs for specific interference with Ngn3 transcript (Baeyens et al., 2006) (Le-sh1Ngn3-eGFP and Le-sh2Ngn3-eGFP) or control sequence (Le-scr-eGFP), immediately followed by PDL. The efficiency of infection was determined 7 days following sham- or virus injection and PDL by direct fluorescence of whole pancreas tail (upper row) and by immunostaining for the reporter on pancreas sections (middle row). The specificity was defined by the fraction of GFP+ cells that immunostained positive for duct cell-specific cytokeratins (47±9%) or the islet cell marker synaptophysin (5±2%). As most acinar cells had disappeared at day 7 following PDL, no GFP+ cells were amylase+ (lower row). (B) As a result of the Ngn3-specific knockdown by Le-shNgn3-eGFP in day 7 PDL pancreas (black bar), the fold activation of Ngn3 was decreased by 70±11% (sh1) and 49±4% (sh2) as compared to the effect of Le-scr-eGFP infection (grey bar, upper). The beta cell mass more than doubled in the tail of Le-scr-infected PDL pancreas compared to sham-operated control (hatched bar) but this effect was inhibited by 66% (sh1) and 26% (sh2) following infection with Le-shNgn3 (middle). Ngn3 knockdown also reduced the increase in the number of insulin+ cells that incorporated BrdU following Le-shNgn3 injection (lower). Abundance of transcripts was quantified by real time RT-PCR using TaqMan probes. Beta cell mass and the fraction of BrdU+ insulin+ cells were determined as in FIG. 1, except that BrdU was supplied 16 and 2 h before sacrifice. All results shown are representative of 3 independent experiments. *: p<0.05 Le-shNgn3-eGFP vs Le-screGFP infected PDL pancreas. Magnification bars are 100 μm.

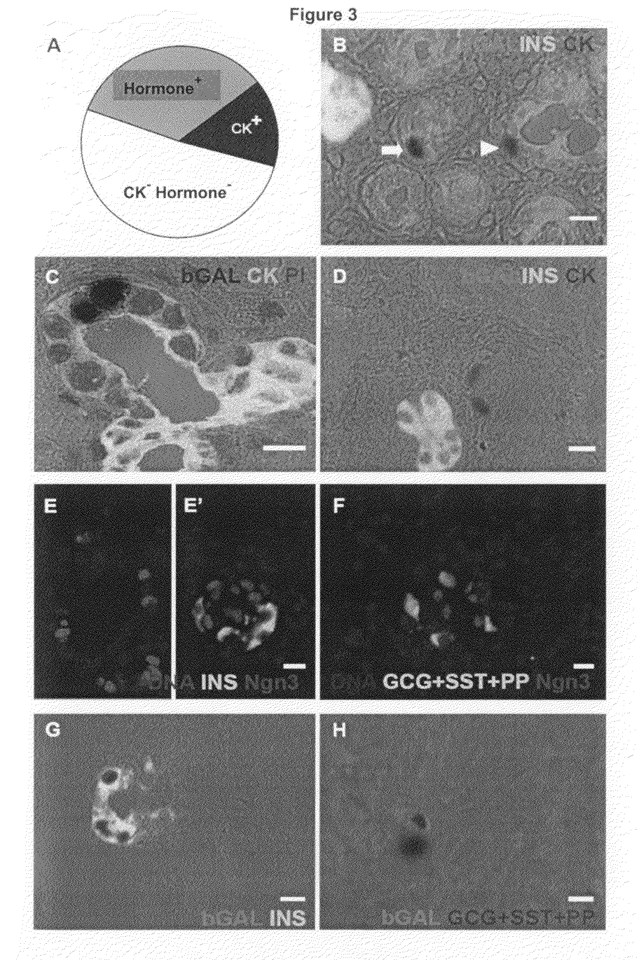

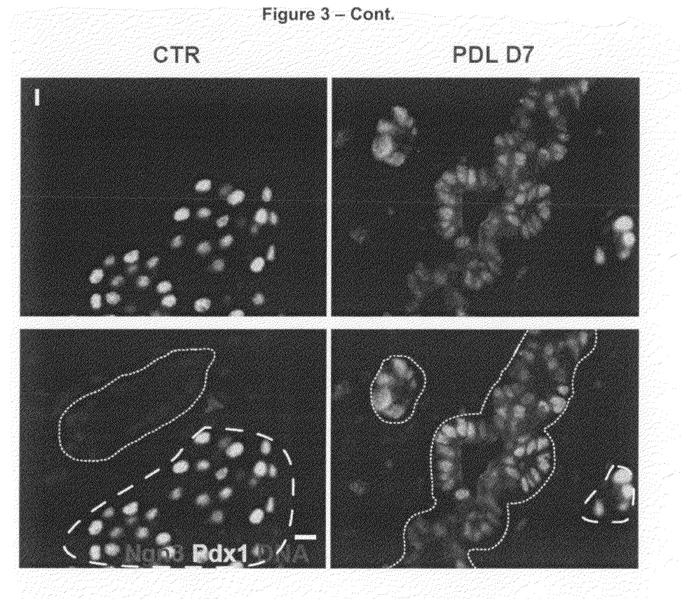

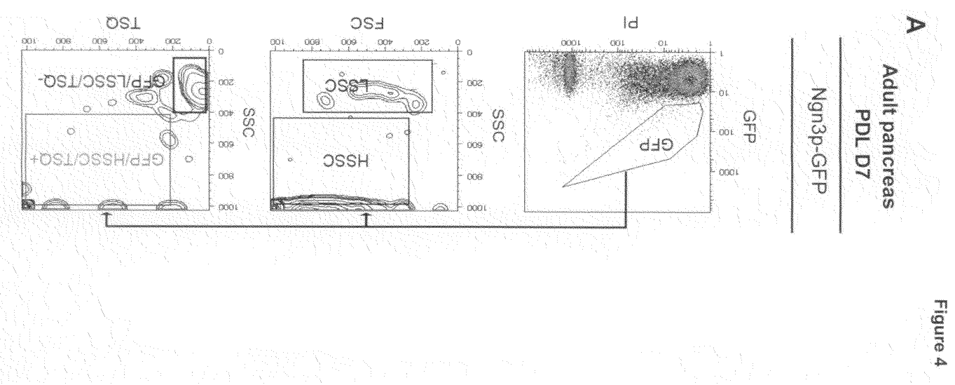

FIG. 3: NEW ISLET CELLS DERIVE FROM HORMONE-PROGENITORS AMONG THE LINING OF DUCTS IN PDL PANCREAS. Duct ligation induced Ngn3 promoter activation in the pancreatic tail of the outbred CD1×C57BU6 strain of Ngn3-nLacZ reporter mice (data not shown), similar as in Balb/c mice. Expression of LacZ, coding for the long-living reporter bGal under the control of Ngn3 promoter sequences, allowed tracking of the Ngn3-expressing cells and their descendants. Ngn3+ cells were detected by histochemical staining of Ngn3-reporter activity (B-D,G,H). The identity of the bGalexpressing cells was determined by combined immunohistochemical detection of ductal cytokeratins and/or islet hormones (B-D,G,H). An overview of the distribution of coexpressing cells was based on the examination of 785 bGal+ cells in the ligated pancreas of 6 mice (A). 15±1% of all bGal+ cells expressed duct cell-specific cytokeratins (CK) but no insulin (INS) (B, arrow) or other islet cell hormones (not shown). Half of the bGal+ CK+ hormone-cells were in direct contact with the duct lumen (C). Half of the bGal+ cells did not express CK or islets markers (here INS) (D), one third of which were in contact with CK+ cells (B, arrowhead). While immunostaining for endogenous Ngn3 showed no co-expression with islet hormones, insulin (E, E′), glucagon (GCG), somatostatin (SST) or pancreatic polypeptide (PP) (F), one third of all bGal+ cells were hormone+ (arrowheads point to double stained cells) indicating that Ngn3+ cells are the source of endocrine islet cells (G,H). DAPI (E-F) and PI (C) stain the nuclei. All sampling was done one week following PDL. No Ngn3 or bGal signal was detected in tail of unligated or head of ligated pancreas. Sham-treated and PDL D7 pancreas were stained simultaneously for Pdx1 and Ngn3 showing co-expression (arrows) in cells lining the duct of PDL pancreas (I). Magnification bars are 10 μm.

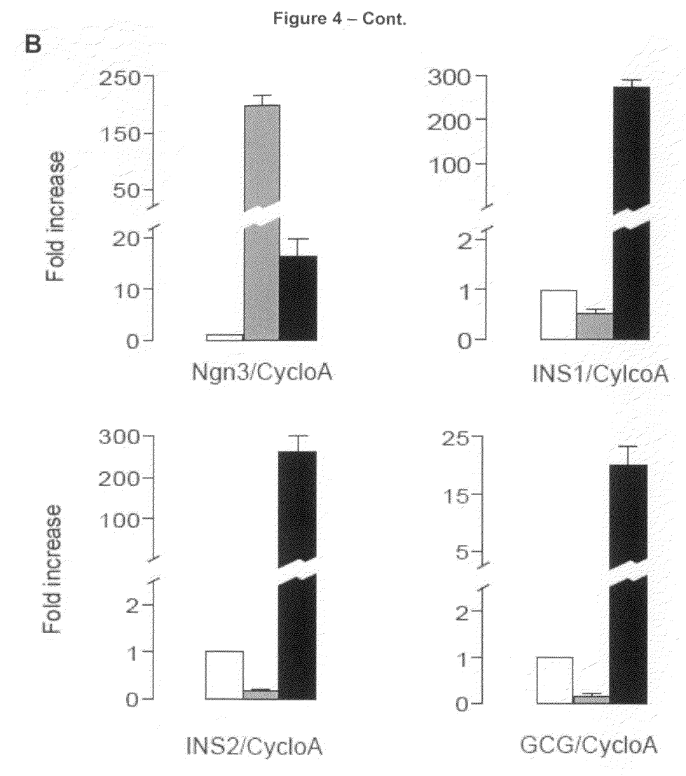

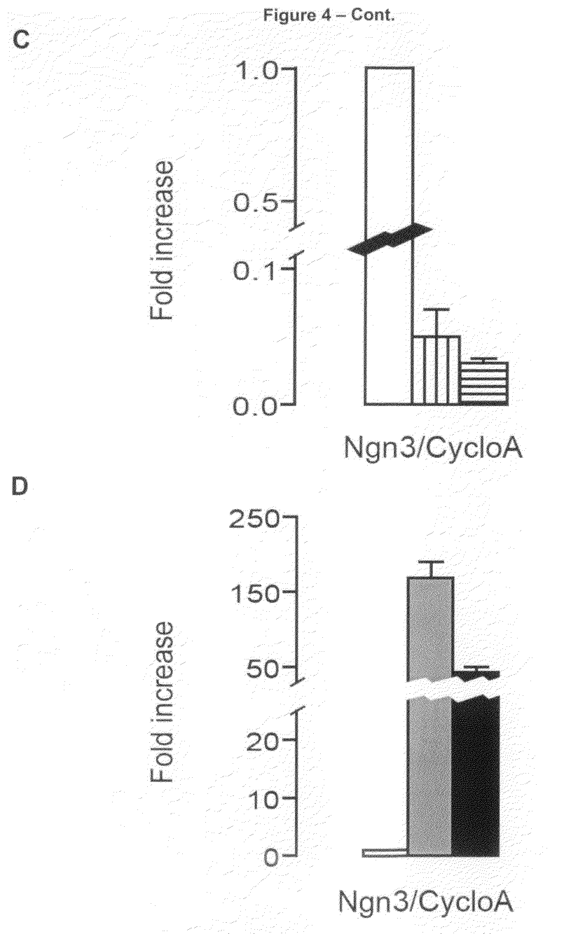

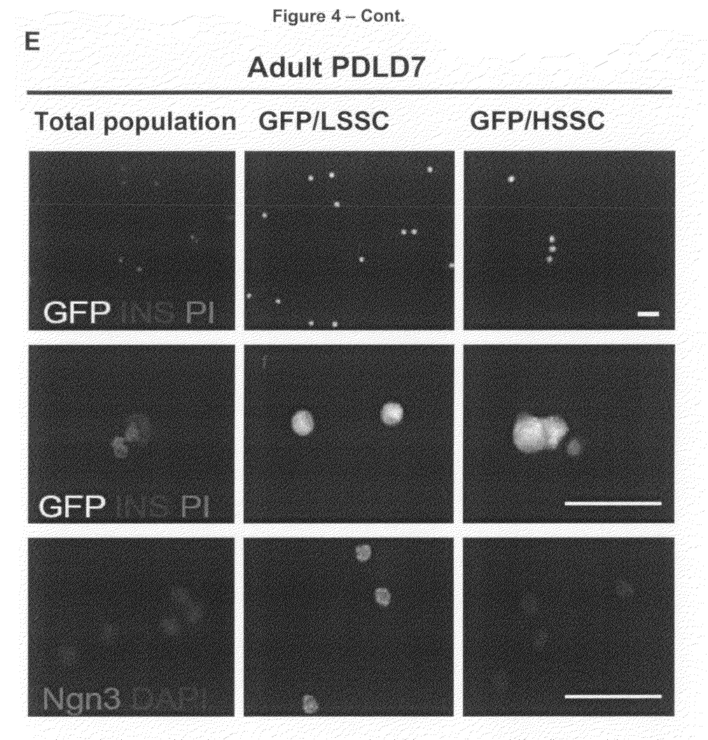

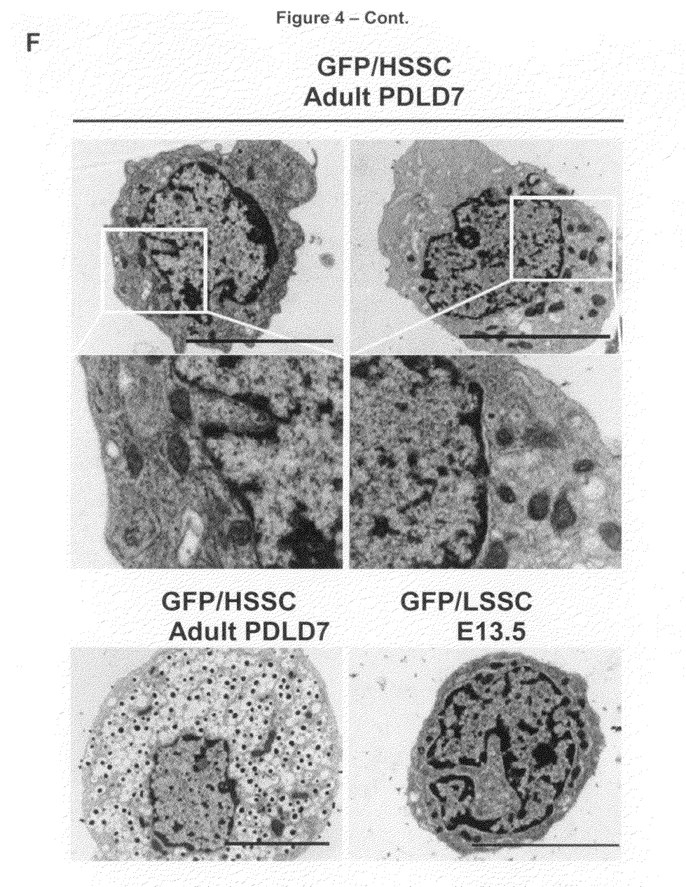

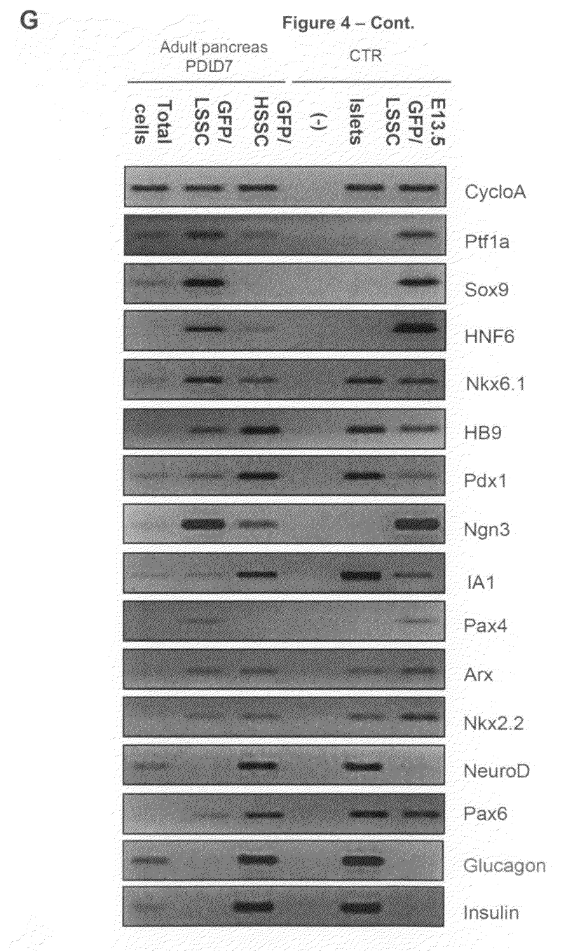

FIG. 4: NGN3+ CELLS ISOLATED FROM ADULT PANCREAS HAVE AN EMBRYONIC ISLET CELL PROGENITOR PHENOTYPE. (A) GFP+ cells were isolated by flow cytometry from adult PDL pancreas (day 7) of Ngn3 reporter mice, based on GFP expression and low degree of granulation. First, viable Ngn3+ cells were isolated based on their GFP-fluorescence and capacity to exclude propidium iodide. Then, they were separated from the hormone+ cells according to their low degree of cellular granulation that was evaluated in two ways, namely by binding of the Zn2+-chelator 6-methoxy-8-p-toluene sulfonamide quinoline (TSQ) to hormone peptides in secretory vesicles and cellular sideward scattering (SSC) properties. The resulting cell population is PI−/GFP+/low SSC/TSQ−, in brief GFP/LSSC (upper window) while granulated GFP+ cells are Pl−/GFP+/high SSC/TSQ+ or GFP/HSSC (lower window). GFP/LSSC cells were not detected in wild-type littermates. (B-D) Quantification of transcript levels by RT-QPCR (see Table S1 and Experimental Procedures). RNA was extracted from the total population of non-sorted pancreas cells (white bars), GFP/LSSC cells (grey bars), GFP/HSSC cells (black bars) and islets from sham-treated (vertical lines) or PDL (horizontal lines) pancreas from transgenic mice with random Ngn3-eGFP insertion (B,C) or eYFP added on the Ngn3 locus (D). All RT-PCR results shown are representative of 3 independent experiments. (E) Immunodetection of insulin+ cells on cytospins of non-sorted and sorted cells from PDL D7 pancreas. Enrichment of GFP+ and Ngn3+ cells and depletion of insulin+ cells in the GFP/LSSC fraction (0 insulin+ on 3000 GFP/LSSC cells, a fraction of the GFP/LSSC cells from 48 PDL mice) was confirmed by immunocytochemistry. Arrowheads in second column point to GFP+ and dapi+ staining, indicating the isolated cells are form a homogenous mixture of NGN3 expressing cells. Arrowheads in the third column show that the HSSC-fraction comprises many insulin-positive cells, whereas the LSSC isolated adult progenitor cells do not. (F) Ultrathin sections of GFP/LSSC (upper panels) and GFP/HSSC cells (lower left panel) from adult PDL (day 7) and of GFP/LSSC cells from E13.5 pancreas (lower right panel) were analyzed on transmission electron micrographs. All cells were isolated from Ngn3-eGFP transgenic mice. Magnification bars are 100 μm in E and 10 μm in F. (G) Compared to non-sorted pancreas cells (Total cells) or GFP/HSSC cells, the expression of progenitor marker Ngn3 and of developmental transcription factors Ptf1a, Sox9, HNF6 and Nkx6.1, located upstream of Ngn3 during embryogenesis, was high in GFP/LSSC cells while that of its direct targets and differentiation markers was low or absent. The presence of transcripts was determined by conventional RT-PCR amplification with specific primers (Experimental Procedures). cDNA from adult mouse islet cells and from GFP+ cells isolated from E13.5 pancreas of Ngn3-GFP reporter mice served as control (CTR). The negative control (−) contained no cDNA.

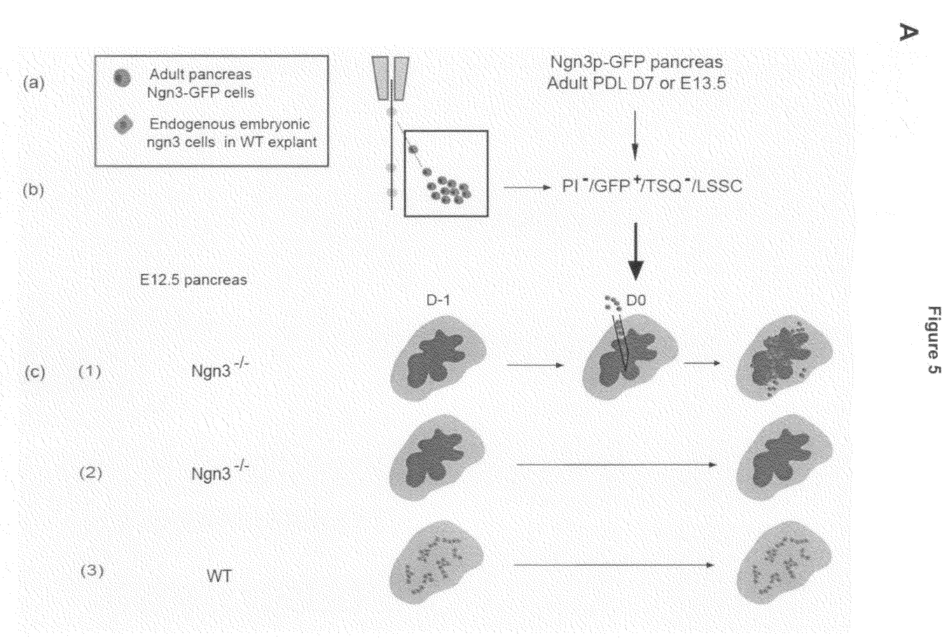

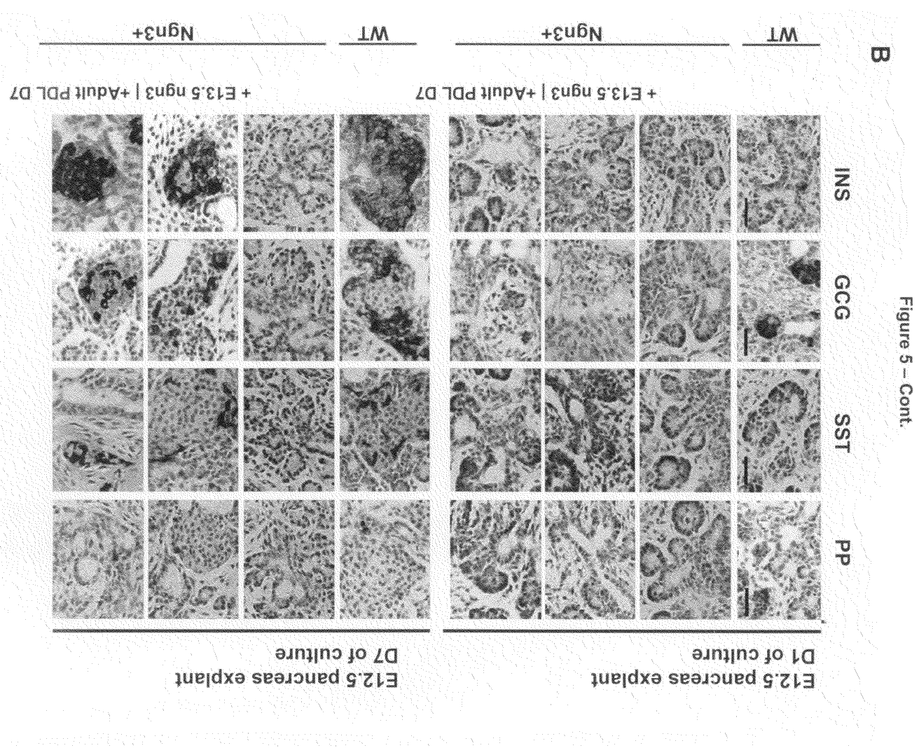

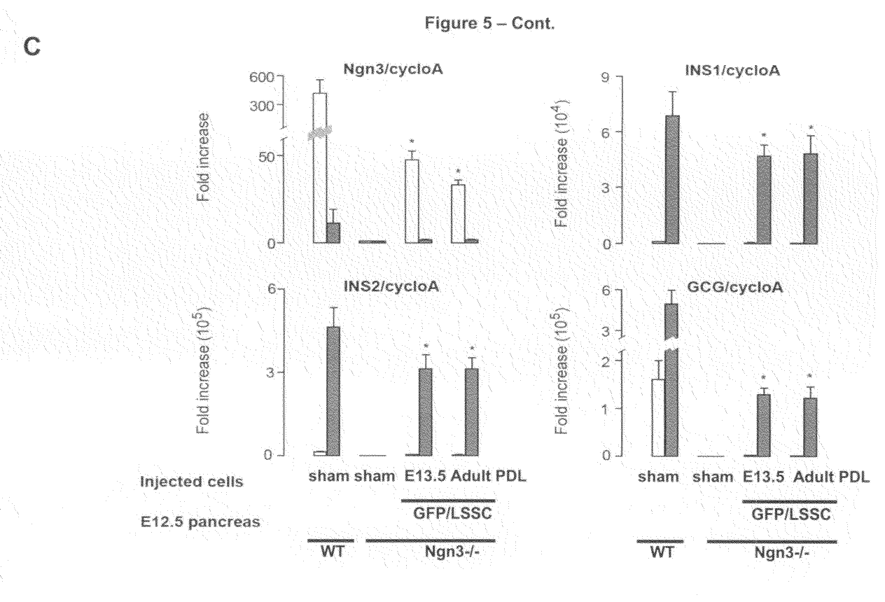

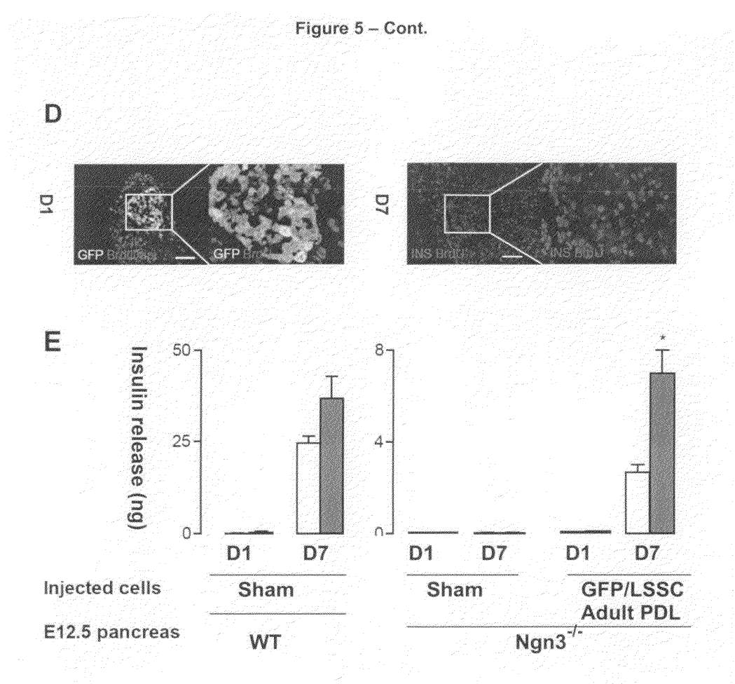

FIG. 5: NGN3+ CELLS FROM ADULT PANCREAS DIFFERENTIATE IN VITRO INTO FUNCTIONAL BETA CELLS. (A) Schematic overview of the experiment: GFP/LSSC cells were isolated by flow cytometry from adult (PDL D7) or embryonic (E13.5) pancreas. Embryonic pancreas was explanted from homozygous Ngn3 null mutant mice or their WT littermates at E12.5 (D−1). One day later (D0), 500 GFP/LSSC cells were micro-injected into the embryonic pancreas and kept in culture for 1 or 7 days. (B) Following 1 day in culture, WT embryonic explants immunostained positive for insulin and glucagon but Ngn3−/− embryonic explants did not, even when injected with GFP/LSSC cells from adult PDL. After one week of culture, WT explants expressed insulin and glucagon, somatostatin and pancreatic polypeptide but Ngn3−/− explants did not. However, when engrafted with GFP/LSSC cells from E13.5 or adult PDL pancreas, the 4 islet hormones were detected in Ngn3−/− explants. Magnification bar is 100 μm. (C) RNA was extracted from the explants described in (B), cultured for 1 (white bar) or 7 (grey bar) days and transcript levels encoding Ngn3, insulin 1 and 2, and glucagon were determined by Quantitative RT-PCR (see Experimental Procedures). The negative control contained no cDNA. (D) While cell cycle activity was high in the explant cultured for 1 day, the engrafted GFP/LSSC cells from adult PDL pancreas were out of cycle. After their differentiation to insulin+ cells, however, the injected cells reinitiated cell cycle. Explants were labeled with BrdU during the last 16 h of culture. (E) The glucose responsive insulin release by embryonic pancreas from Ngn3−/− mice engrafted with GFP/LSSC cells was determined at day 1 and day 7 of culture, following incubation in 6 mmol/L (white bars) or 20 mmol/L (grey bars) glucose for 24 hours. Explants from embryonic pancreas of WT mice and of non-engrafted Ngn3−/− mice were taken as positive and negative control, respectively. All results shown are representative of 3 independent experiments. *: p<0.001 insulin release at 6 mmol/L vs 20 mmol/L glucose.

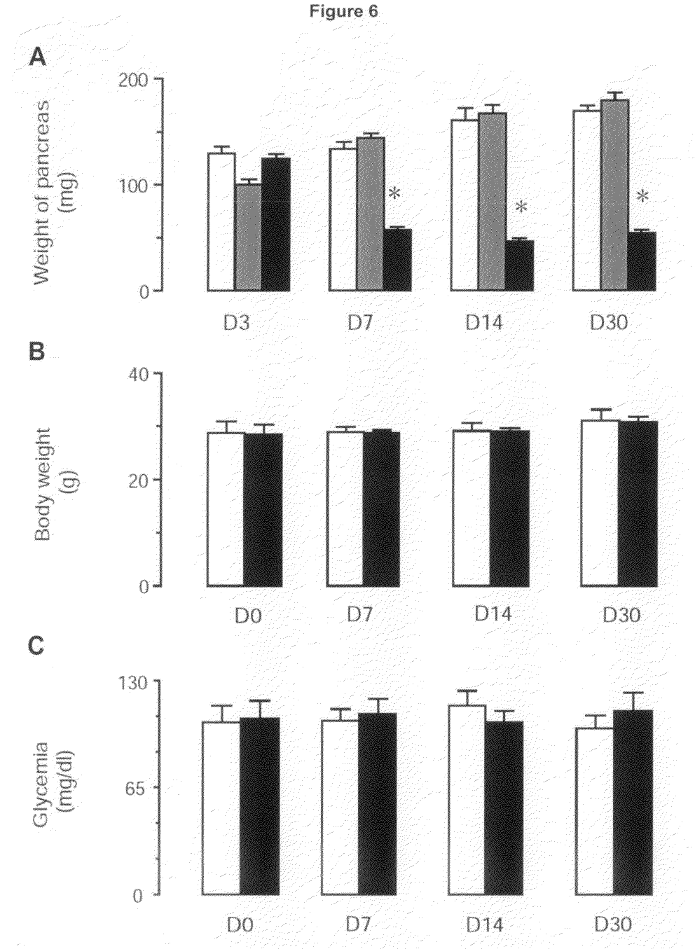

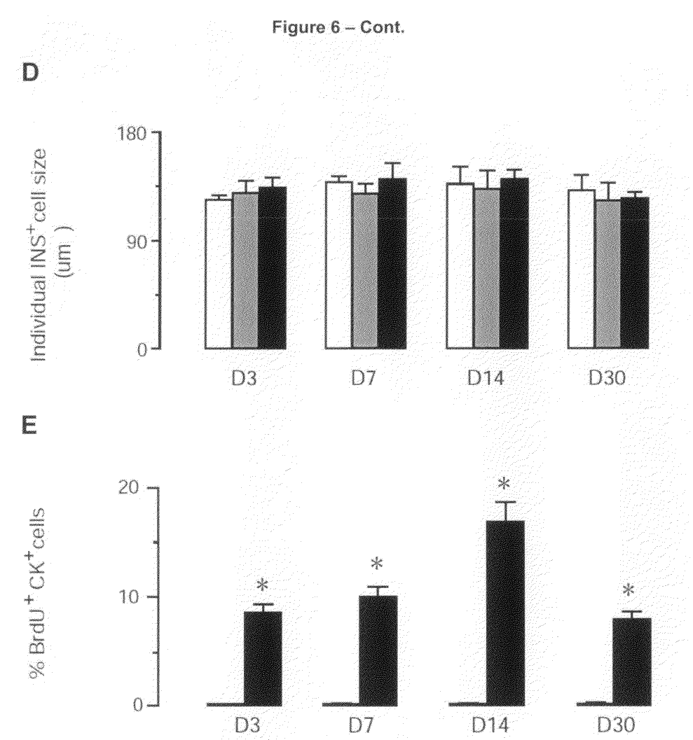

FIG. 6: PDL AFFECTS THE WEIGHT OF THE PANCREATIC TAIL AND DUCT CELL PROLIFERATION BUT NOT MOUSE BODY WEIGHT, GLYCEMIA OR INDIVIDUAL INSULIN+ CELL SIZE. From sham-operated (white bars) and PDL mice (black bars) body weight (g) (A) and glycemia (mg/dl) (B), were measured at different time points (day 0, 7, 14 and 30) following surgery. The weight (mg) (C), individual size of beta cells (μm) (D) and BrdU incorporation in duct cells (% BrdU+CK+ cells, BrdU injection of 50 mg/kg mouse BW, one hour before sacrifice) (E) from the tail of sham-treated pancreas (white bars) and head (grey bars) or tail (black bars) of PDL pancreas were determined at day 3, 7, 14 and 30 following surgery. All results shown represent at least 6 independent experiments. *: p<0.001, tail from ligated vs unligated pancreas.

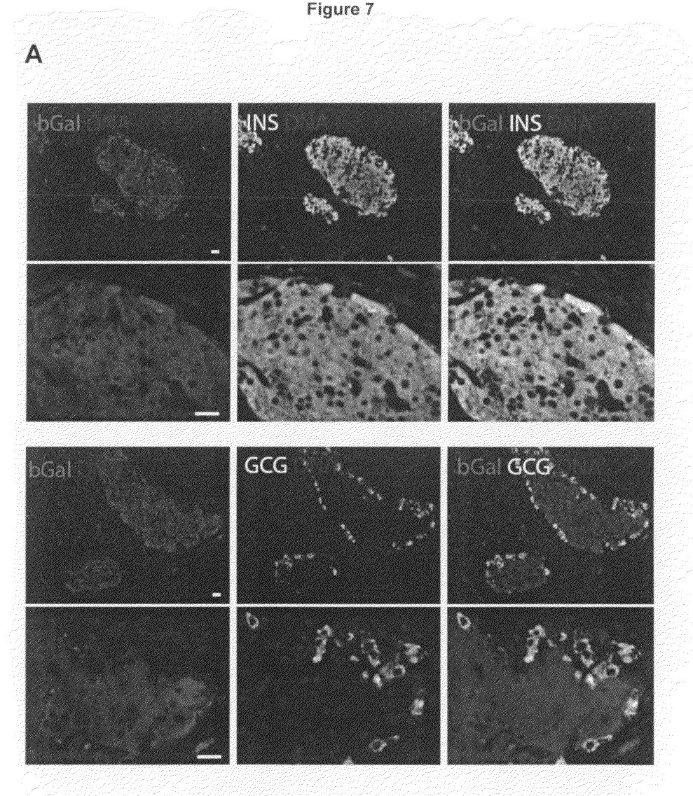

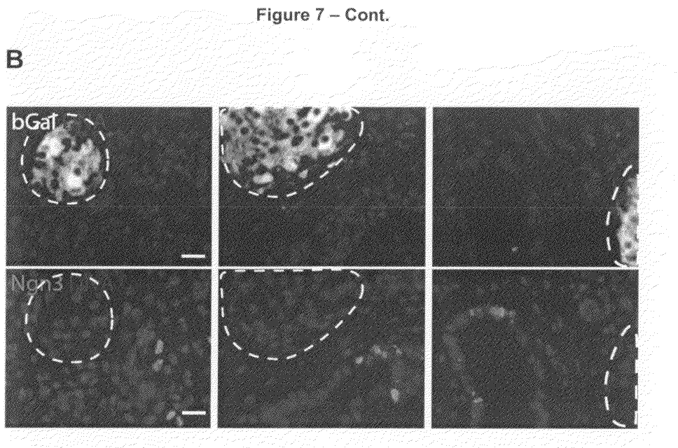

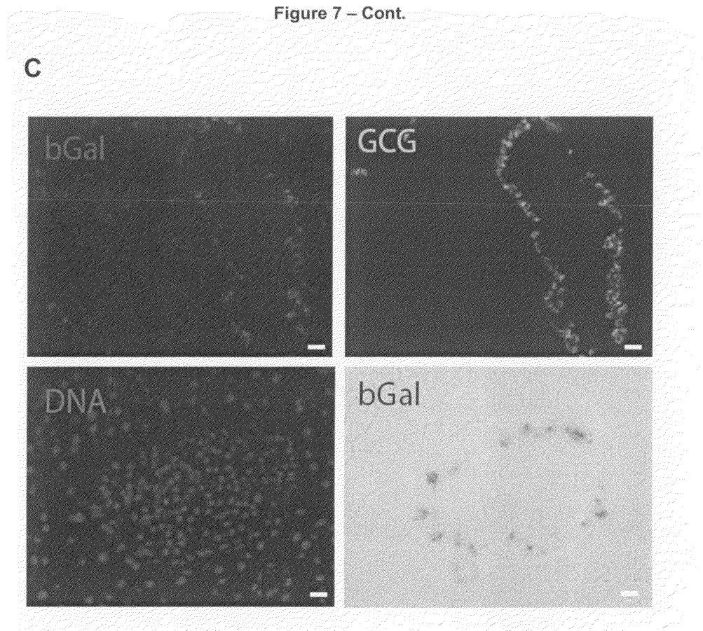

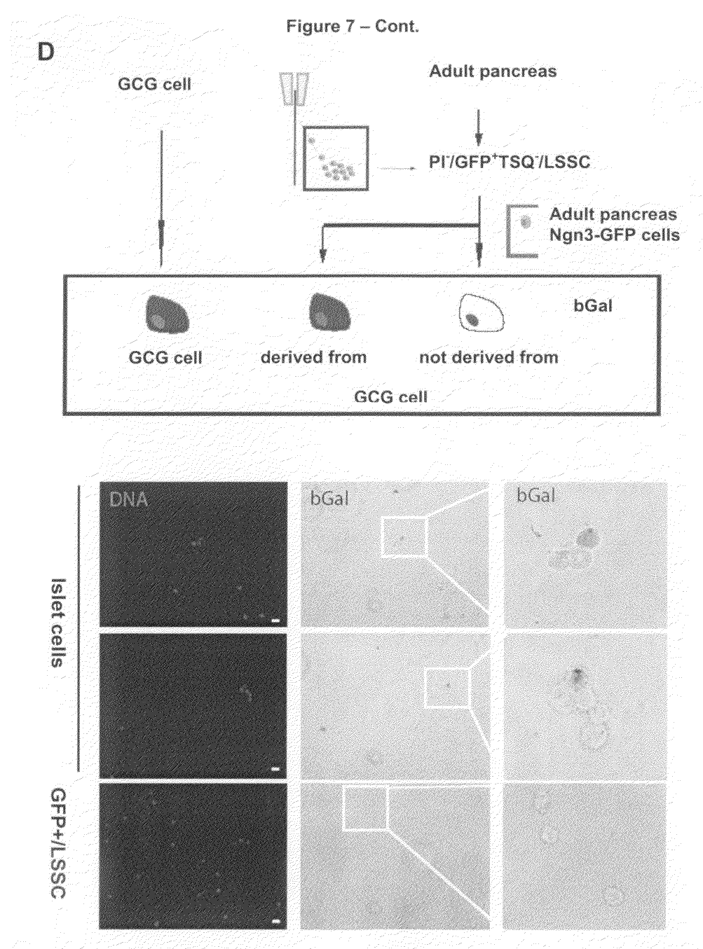

FIG. 7: NGN3-ENCODING CELLS DO NOT DERIVE FROM DEDIFFERENTIATING ISLET CELLS. INS-Cre/R26R and Ngn3-eGFP/GCG-Cre/R26R mice permanently express the reporter bGal under control of the constitutively active promoter, ROSA26, in insulin (INS)+ (A) and glucagon (GCG)+ (C) cells, respectively. In PDL pancreas of INS-Cre/R26R mice no Ngn3+ cells were bGal+ (B) demonstrating that Ngn3+ cells did not derive from insulin+ cells. GFP/LSSC cells were sorted from the ligated pancreas tail of Ngn3-eGFP/GCG-Cre/R26R mice. On a total of >500 sorted GFP/LSSC cells from PDL pancreas of Ngn3-eGFP/GCG-Cre/R26R mice none were bGal+, while 31 cells were bGal+ on 620 isolated islet cells, demonstrating that none of the sorted GFP/LSSC cells expressed glucagon earlier in life (D). Magnification bars are 10 μm.

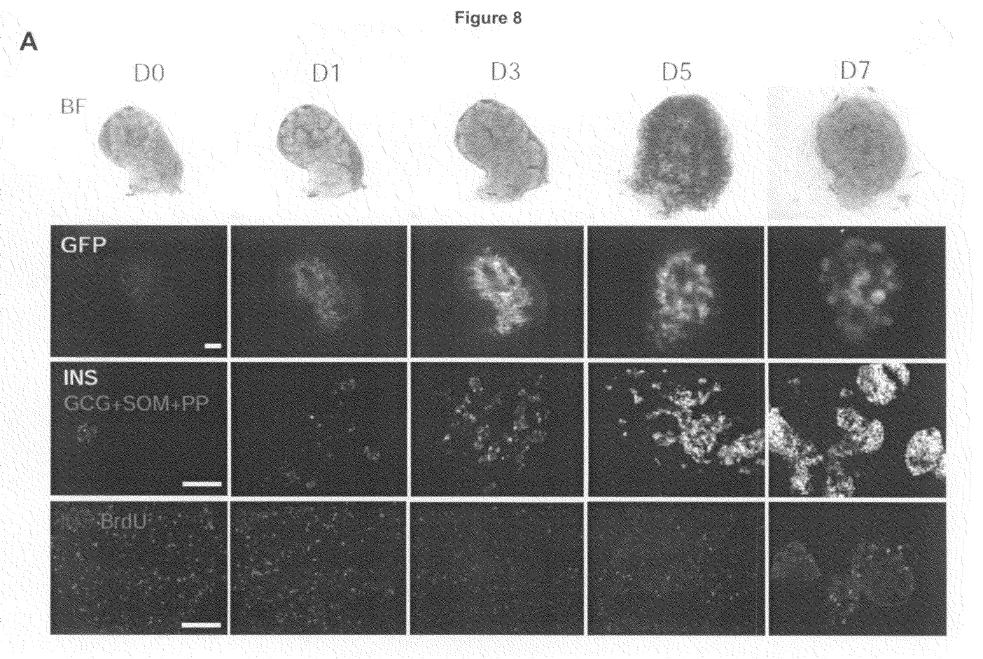

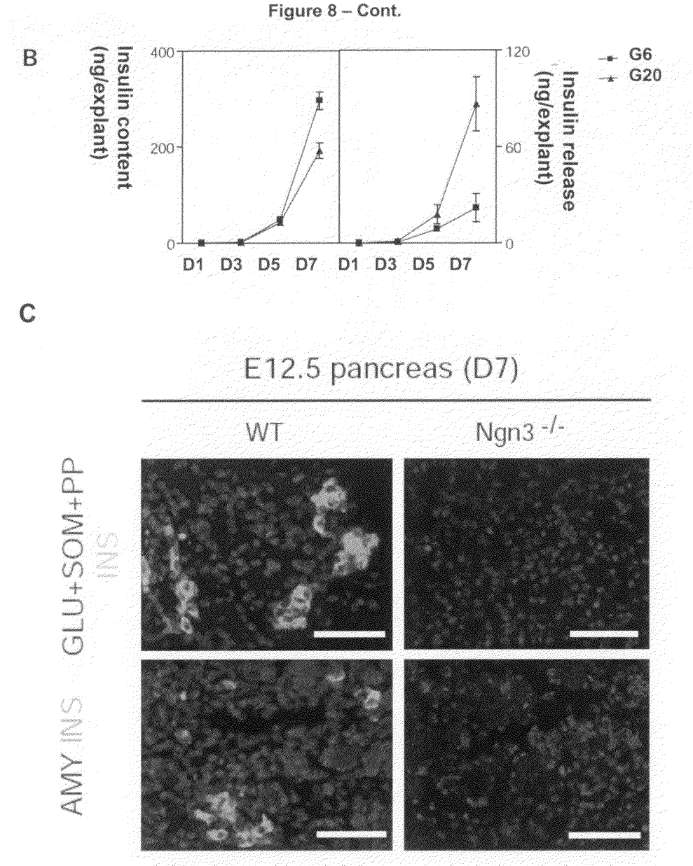

FIG. 8: EXPLANTED EMBRYONIC PANCREAS DIFFERENTIATES IN CULTURE. E12.5 embryonic pancreases were isolated from Ngn3p-eGFP reporter (rows 1 and 2) or Balb/c mice (rows 3 and 4) at day 0 (D0) and cultured for 7 days (Miralles et al., 1998). Time-dependent changes in the explant morphology (row 1: phase contrast microscopy; BF: brightfield), eGFP reporter expression (row 2: direct fluorescence), islet cell hormones (row 3) and beta cells with active cell cycle (row 4, explants were labeled with BrdU during the last 2 h of culture) (A). The insulin content of both the explant (left panel) and the culture medium (right panel) increased time-dependently (B). No endocrine hormones (insulin-specific immunostaining and combined glucagon, somatostatin and pancreatic polypeptide staining using a mix of 3 antibodies) were expressed in homozygous Ngn3-null mutant mouse pancreas while acinar cell differentiation, as determined by amylase-specific immunostaining, was normal (C). Magnification bars are 100 μm (A,C).

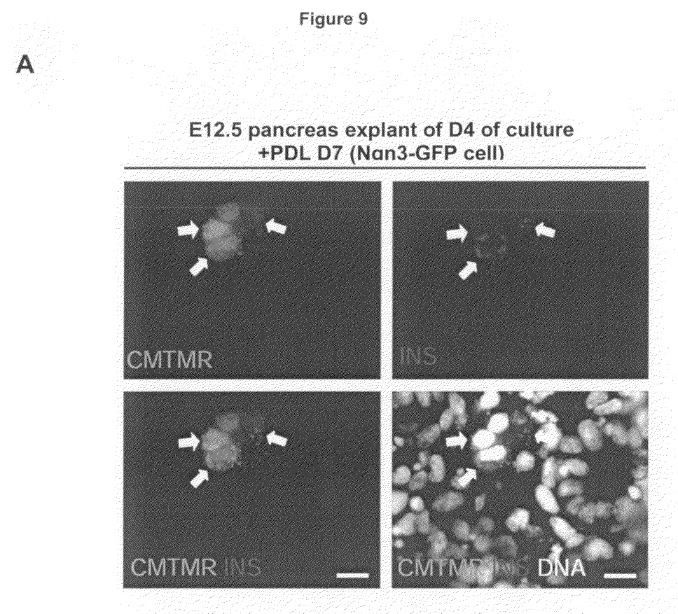

FIG. 9: ENDOCRINE DIFFERENTIATION OF GFP/LSSC CELLS IS AUTONOMOUS. GFP/LSSC cells were labeled by pre-incubation with CellTracker Orange CMTMR and labeled cells could be traced until 4 days after injection in the explanted pancreas of Ngn3−/− embryo's. Some of the CMTMR-labeled cells already expressed insulin at this early stage of differentiation. Magnification bars are 10 μm.

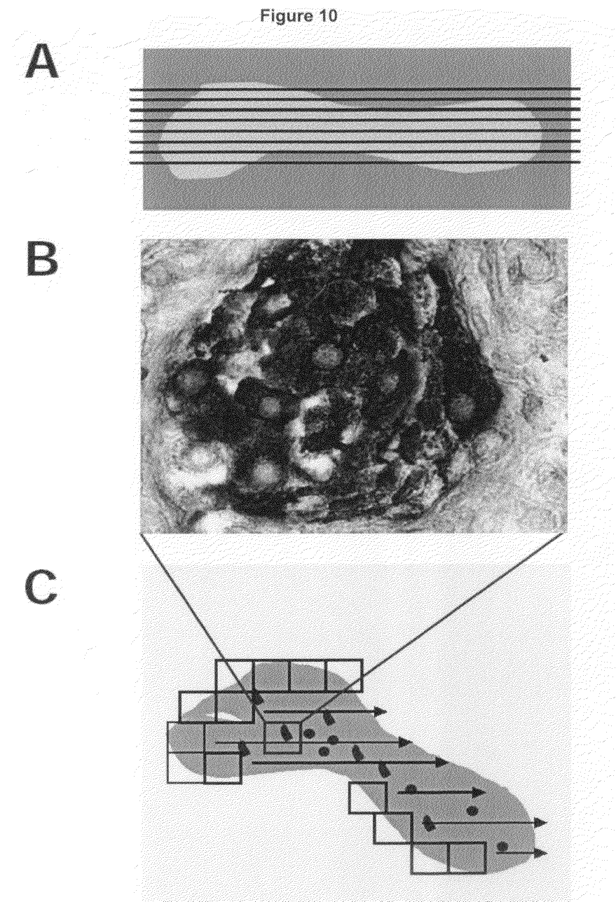

FIG. 10: METHOD FOR QUANTIFICATION OF BETA CELL MASS AND PROLIFERATION. Pancreas was dissociated and its weight was determined on a microbalance. The tissue was fixed (4 hours in 4% FA) and embedded in paraffin. The whole organ was sectioned (A). We examined 9 tissue sections of 5 μm, taken at 150 μm distance, and (immuno) stained for hematoxilin, insulin (AP) and BrdU (DAB) (B). Pictures were taken at 20-fold magnification and compiled to represent the complete area of the section (C). The insulin+ area as well as the total, i.e. hematoxilin-stained, area was manually encircled and the surfaces of both encircled areas were measured using NIH ImageJ (vs. 1.3.1) software. The beta cell mass=(insulin+ area/area of the pancreas section)×pancreas weight. The numbers of BrdU+ nuclei and insulin+ cells were counted manually. Percentage of BrdU+ insulin+ cells and the individual insulin+ cell area were calculated.

### DETAILED DESCRIPTION OF THE INVENTION

The present invention demonstrate (cf. example 1) convincingly that the adult mouse pancreas contains islet cell progenitors and that expansion of the beta cell mass following injury induced by ligation of the pancreatic duct depends at least partly on the activation of Ngn3 gene expression and the ensuing differentiation of endogenous progenitor cells in a cell autonomous, fusion-independent manner. Partial duct ligation induces a strong inflammatory response and a loss of acinar cells. Both processes may be important in signaling for increase of the beta cell mass under these conditions of injury but it is unclear at this moment whether they play a role in the normal physiology of a healthy pancreas where the importance of self-duplication rather than stem cell differentiation is well documented (Dor et al., Nature 429:41-46, 2004; Teta et al., Dev. Cell 12:817-826, 2007). Activation of Ngn3 and doubling of the beta cell mass could be prevented up to 66% by Ngn3-specific RNA interference, suggesting a considerable contribution of progenitor cells to the observed beta cell hyperplasia. In non-ligated pancreas 67% of the cells were transduced compared to only 18% in ligated pancreas. This difference is due to the disappearance of acinar cells, the most abundant cell type of the infected pancreas and to a massive recruitment of uninfected immune response cells to the pancreas affected by inflammation following PDL. The efficient Ngn3 knockdown can be explained by (i) the infection of 67% of pancreas cells before PDL is carried out, (ii) the specific location of an important fraction of Ngn3-expressing cells, targets of the interfering RNA, among or in contact with duct cells that line the site of injection and therefore are exposed directly to the virus and (iii) a near 100% knockdown of Ngn3 expression by Le-sh1Ngn3. The remaining increase in beta cell mass in spite of Ngn3 knockdown likely is due to cycling of (i) pre-existing beta cells and/or (ii) progenitor cells that were uninfected or that had differentiated beyond the Ngn3+ stage before being infected. While under normal physiological conditions the slow course of beta cell proliferation is sufficient to compensate for their low turn over and expansion (Dor et al., Nature 429:41-46, 2004; Teta et al., Dev. Cell 12:817-826, 2007), our data in injured tissue demonstrate a rapid course of hyperplasia that depends on progenitor cell recruitment. This pathway may not be active after 50-70% partial pancreatectomy (PPx) (Dor et al., Nature 429:41-46, 2004; Teta et al., Dev. Cell 12:817-826, 2007), a less robust injury model in which Ngn3-expressing cells remain absent (Lee et al., Diabetes 55:269-272, 2006) and the beta cell mass indeed increases much slower than following duct ligation (Bouwens and Rooman, Physiol. Rev. 85:1255-1270, 2005). The Ngn3+ islet cell progenitors co-express cytokeratins when located among the cells that line the pancreatic ducts and were activated by PDL as shown by expression of Pdx1. In PDL pancreas, the Ngn3+ cells near and within islets did not express any of the islet cell hormones, nor did permanently labeled islet cells express Ngn3. Finally, islets isolated from PDL pancreas contained less Ngn3 mRNA than total PDL pancreas, excluding dedifferentiation of pre-existing islet cells as the basis of the phenomena we describe. The detection of bGal in the progeny of Ngn3+ cells that already expressed the hormones, some of which were in islets, suggests a migration from duct-to-islet by the progenitor cells. The ultrastructure of Ngn3+ cells from adult pancreas revealed an immature phenotype but when injected in an embryonic microenvironment that supports islet progenitor differentiation, the GFP/LSSC cells became functional endocrine islet cells among which were beta cells with glucose responsive insulin release. We confirmed that the 6 kb promoter recapitulates the endogenous Ngn3 expression by performing PDL on the pancreas of Ngn3eYFP/+ knock-add-on mice (Mellitzer et al, Mol. Endocrinol. 18:2765-2776, 2004) and showing that YFP/LSSC cells are similar to the GFP/LSSC cells found in PDL pancreas from Ngn3-GFP mice. The endogenous progenitor cell type we isolated from adult mouse pancreas is different from the atypical ones isolated from neonatal or adult mouse pancreas that expressed Ngn3 but had a high proliferation capacity and gave rise to pancreatic and neuronal cell types in vitro (Seaberg et al., Nat. Biotechnol. 22:1115-1124, 2004; Suzuki et al., Gene Ther. 10:15-23, 2004). None of these expanded colonies formed islet cells with significant glucose responsive insulin release. Recently, the non-endocrine fraction of the human pancreas, containing undifferentiated epithelial cells that expressed markers of pancreatic duct cells was used to generate new insulin-producing cells when grafted together with cells of fetal pancreas under the kidney capsule of mice (Hao et al., Nat. Med. 12:310-316, 2006). An important similarity with our study is the requirement of an embryonic microenvironment able to produce essential growth and differentiation factors. Cytokeratin 19, the marker used by Hao et al. (Hao et al., Nat. Med. 12:310-316, 2006), is ambiguous though, since it is expressed in islet cells undergoing dedifferentiation (Gao et al., Diabetologica 48:2296-2304, 2005). Ngn3, however, is the only unambiguous marker known for islet progenitors in the embryonic (Gu et al., Development 129; 2447-2457, 2002) and in the adult pancreas (present study). Our data provide the first direct evidence for the existence of endogenous endocrine islet cell progenitors in adult mouse pancreas. This cell population is similar to the one that gives rise to the islets during embryonic development and represents an obvious target for therapeutic regeneration of beta cells in diabetes. Indeed, our findings reveal the significance to investigate the feasibility of (i) isolating facultative beta cell progenitors and newly formed beta cells from human pancreas in order to expand and differentiate them in vitro and transplant them in diabetic patients and (ii) composing a mix of factors able to activate beta cell progenitors to expand and differentiate in situ in patients with an absolute or relative deficiency in insulin.

Methods for isolating adult islet progenitor cells and cells obtained thereby:

In a first aspect the present invention relates to a method for preparing pancreatic islet (e.g. alpha-, beta-, delta-, or pp-) cells starting from islet progenitor cells comprising the steps of:
a) isolating progenitor cells from the pancreas of an adult mammal, by the steps of
   i) providing transgenic mammals, preferably animals such as rodents, expressing a reporter gene under the control of a suitable promoter, preferably an alpha-, beta-, delta-, or pp-cell specific promoter,
   ii) detecting the presence of said progenitor cells by detecting the product of said reporter gene, and
   iii) isolating the progenitor cells detected in step ii),
b) inducing differentiation of said isolated progenitor cells into differentiated pancreatic islet cells, and
c) proliferating the differentiated pancreatic islet cells obtained in step b).

In one embodiment, steps b) and c) are carried out in an explanted embryonic pancreas environment from a non-human mammal. In another embodiment, steps b) and c) are done in an in vitro cell culturing system, by addition of the necessary regulatory factors identified by the methods defined below.

In accordance with the method of the present invention, said progenitor cells are preferably isolated using a progenitor cell specific marker. An example of such a progenitor cell specific marker is the Ngn3 marker. In a preferred embodiment, the said promoter is a progenitor cell-specific promoter. An example of such a progenitor cell-specific promoter is the Ngn3 promoter.

Preferably, in accordance with the present method said progenitor cells originate from non-endocrine cells cells, lining the pancreatic duct.

In another aspect, the invention provides islet progenitor cells, e.g. beta progenitor cells, obtainable by carrying out step a) of the method according to the invention. Present progenitor cells are characterized in that said cells express the Ngn3 functional marker gene.

In yet another aspect the invention provides pancreatic islet cells, among which are alpha-cells, beta-cells, delta-cells and pp-cells that are obtainable by carrying out the method according to the present invention.

In accordance with the present invention A) the (pancreatic) islet cells, such as beta-cells, B); the progenitor cells, such as beta-cell progenitors, and/or C) the explanted embryonic pancreas according to the invention also find numerous applications in the research strategies. In another aspect, the present invention also relates to the use of the Ngn3 marker gene for diagnostic and/or research purposes. For instance of the Ngn3 gene can be used in accordance with the present invention as a marker gene for identifying antigenic determinants of the islet progenitor cells, preferably for identifying antigenic determinants that are exposed towards the external surface of the cells. The present invention also encompasses the generation of antibodies against these external antigenic determinants. Generation of antibodies against these external antigenic determinants allows isolating islet (e.g. alpha-, beta-, delta- or pp-) progenitor cells also from human pancreas, and to differentiate and expand these cells in vitro before syngeneic transplantation.

Identifying antigenic determinants that are specific for adult islet cell progenitors:

In an alternative embodiment, the invention provides for the use of progenitor cells, as defined herein to identify antigenic determinants that are characteristic for a type of progenitor cells, e.g. characteristic for beta cells. The term “antigenic determinant” as used herein refers to the epitope of an antigen, the immunologically active region of an immunogen that binds to antigen-specific membrane receptors on lymphocytes or to secreted antibodies. An antigenic determinant refers to that part of an antigenic molecule against which a particular immune response can be directed. Such antigenic determinants can advantageously be applied during the isolation process of progenitor cells, e.g. when isolating progenitor cells from different mammals, including humans.

A typical method for identifying antigenic determinants that are specific for adult islet cell progenitors of the invention, comprises the step of:

i) analyzing the transcriptome of an adult islet progenitor cell

ii) comparing the transcriptome of i) to the transcriptome of an embryonic islet (e.g. beta-) cell progenitor of the same stage, and

(iii) comparing the transcriptome of i) to the transcriptome of an adult differentiated beta cell, thereby identifying factors or markers that are specific for adult beta cell progenitors, wherein the progenitor cells are alpha-cell, beta-cell, delta-cell or PP-cell progenitor cells.

The antigenic determinants identified with this method can be used in a method for identifying binding molecules (e.g. antibodies) that specifically recognize surface antigens on adult pancreatic islet progenitor cells. Such binding molecules (e.g. antibodies) can be used for the isolation of adult islet cell progenitors in a subject, preferably a human subject. Again, the progenitor cells are alpha-cell, beta-cell, delta-cell or PP-cell progenitor cells.

Isolation of islet progenitor cells:

Furthermore, the invention provides for the isolation of the islet (e.g. alpha-, beta-, delta- or pp-) progenitor cells from adult human pancreas using binding molecules that specifically recognize surface antigens defined by the above mentioned transcriptome analysis.

The method for isolating adult islet progenitor cells can for example be performed by separating labelled cells from non-labelled cells using standard separation techniques based on the retention of labelled binding molecules directed to one or more of the biomarkers of the present invention. One option is to use antibodies, aptamers or other specific binding agents or ligands, directed to one of the biomarkers of the invention, for tagging cells of interest with a small magnetic particle or magnetic bead. The bead-binding molecule conjugate is then directed to the beta-cell progenitors in the pancreatic cell preparation and the beta cell progenitors can be specifically purified from the total pancreatic cell preparation by using e.g. an electromagnetic field. In some systems, the sample is processed through a column that generates a magnetic field when placed within the separator instrument, retaining only the labeled cells. Other systems offer simplified versions of the magnetic separator. Instead of a column and separator instrument, these systems use a simple magnet to directly retain the labeled cells within the tube, while the supernatant is drawn off. Some of these systems can be used in a positive or negative selection manner. Negative or enrichment selection means that unwanted cells can be labeled (captured), leaving the cells of interest label-free. The magnetic particles do not interfere with flow cytometry, nor do they interfere with cell growth, according to, so cells that have been isolated using such a system can be further cultured. Magnetic separation has proven uniquely powerful and broadly applicable, sometimes leading to 70% recovery of the target cells and up to 98% purity while retaining cell viability. Alternatively, an efficient non-magnetic separation method, based on work on tetrameric antibody complexes (TACs) works by linking unwanted cells in a sample together, forming clumps. After labeling, the sample is layered over a buoyant density medium such as Ficoll. The labeled cells pellet with when centrifuged, while the desired, unlabeled cells are recovered at the interface. This method is fast and the cells obtained are not labeled with antibodies and are untouched. FAGS-sorting can also be used, as is shown in FIG. 4 of example 1 further down.

In an alternative approach, the isolated adult islet progenitor cells of the invention are administered to an animal, in the presence or absence of an adjuvant, in order to elicit an immune response in the animal. Blood samples obtained from the animal in regular time frames will be analyzed (e.g. through an ELISA system or the like) for the presence or absence of antibodies. Obtained antibody-comprising samples can be further purified and selected for specificity and/or affinity towards adult islet cell progenitors. Preferred animals for use in the immunization experiments are camelids such as camel, dromedary and llama for the isolation of single chain antibodies or mice, rabbits and rats for the isolation of monoclonal or polyclonal antibodies, using technologies known in the art.

The term “pancreatic islet progenitors or islet progenitor cells” is used herein to denote cells that are able to differentiate to one of the four types of pancreatic islet cells, i.e. alpha-cells, beta-cells, delta-cells or PP-cells. The term “beta cell progenitor or beta cell progenitor cells” is used herein to denote cells that are able to differentiate to pancreatic beta-cells.

The term “binding molecules” used in the methods and kits of the invention refers to all suitable binding molecules that are specifically binding or interacting with one of the biomarkers of the invention and that can be used in the methods and kits of the present invention. Examples of suitable binding agents are antibodies, aptamers, specifically interacting small molecules, specifically interacting proteins, and other molecules that specifically bind to one of the biomarkers. Both monoclonal, polyclonal or single chain antibodies or fragments thereof that bind one of the biomarkers of the present invention are useful in the methods and kits of the present invention. The monoclonal and polyclonal antibodies can be prepared by methods known in the art and are often commercially available. Aptamers that bind specifically to the biomarkers of the invention can be obtained using the so called SELEX or Systematic Evolution of Ligands by EXponential enrichment. In this system, multiple rounds of selection and amplification can be used to select for DNA or RNA molecules with high specificity for a target of choice, developed by Larry Gold and coworkers and described in U.S. Pat. No. 6,329,145. Recently a more refined method of designing co-called photoaptamers with even higher specificity has been described in U.S. Pat. No. 6,458,539 by the group of Larry Gold. Methods of identifying binding agents such as interacting proteins and small molecules are also known in the art. Examples are two-hybrid analysis, immunoprecipitation methods and the like.

Identification of regulatory factors important for the development and differentiation of progenitor islet cells into functional islet cells:

In a further embodiment, the invention provides for a method for identifying extracellular factors such as growth factors, cytokines, chemokines or other regulating factors needed for the induction of differentiation of islet progenitor cells in vivo. Such factors can be used in a therapeutic application for the activation of the differentiation of progenitor beta cells into mature beta cells, capable of producing insulin. Such an approach would seriously improve the ability of treating islet cell related disorders, since no transplantation or injection of islets or islet cells would be needed, only a treatment regime with the relevant regulating or differentiating factors would suffice.

The invention therefore provides for a method for identifying the regulatory factors for the differentiation of islet cell progenitors into biologically active islet (e.g. alpha-, beta-, delta- or pp-) cells comprising the steps of:

a) isolating islet (e.g. alpha-, beta-, delta- or pp-) progenitor cells from the pancreas of an adult mammal, by the steps of:
i) providing transgenic mammals, preferably animals such as rodents, expressing a reporter gene under the control of a suitable promoter,
ii) detecting the presence of said progenitor cells by detecting the product of said reporter gene, and
iii) isolating the islet progenitor cells detected in step ii), and

b) culturing the cells obtained in step a) in the presence or absence of regulatory factors

c) Monitoring differentiation of the progenitor cells into functional beta cells in order to identify those factors needed for differentiation of progenitor cells into functional pancreatic islet cells.

The regulatory factors for use in the methods and compositions of the invention are preferably selected from the groups of chemokines, cytokines, hormones, steroids, survival factors, transmitters, growth factors, proliferation factors and/or differentiation factors. Preferable, the regulatory factors are selected from the group of IGF1/2 or BPs, MCP, IL1, IL6 (LIF, CNTF), IL12, IL18, CXCL1, CXCL15, IFNgamma, TNFalpha/beta, GLP1/2, GIP, glucagon, insulin, somatostatin, gastrin, ghrelin, leptin, PYY, NPY, CCK, fractalkine, HB-EGF, betacellulin, amphiregulin, HGF/SF, NGF, midkine1, pleiotrophin, HDGF, VEGF, PIGF, KITL(SCF), G/M-CSF, FGF, HPL, GMFG, Wnt, TCF, APC, TGFbeta, activinA/B, BMP2/5/6/7/8, nodal, CTGF, GDF1/LASS1, contactin, jagged1, HH, netrin, neogenin, osteopontin, osteoglycin, SDF1, TLR ligands and steroids.

Any one of the regulatory factors mentioned above can be used alone or in combination with any one or more other regulatory factors.

The invention provides for the analysis of the full transcriptome of the adult islet (e.g. alpha-, beta-, delta- or pp-) progenitor cell by comparison with (i) embryonic progenitors of the same stage and (ii) fully differentiated corresponding islet cells to define known and unknown signaling factors that determine the status of activation and differentiation of the islet progenitor cells.

In addition, the invention provides for the analysis of the proteome of the infiltrating cells of the immune system to characterize the signals that determine the status of activation and differentiation of the progenitor cells. Focus on cytokines, chemokines, proliferation and differentiation factors. In one such embodiment, regulatory factors such as the ones indicated above comprising cytokines, chemokines, growth- and differentiation factors are analyzed for their presence in infiltrating myeloid (CD11B) cells using xMAP technology (Luminex), quantitative PCR-technology, ELISA-technology or micro-array technology in order to identify all regulatory factors involved in the differentiation process.

In a following experiment, the differentiation of adult islet progenitor cells will be simulated by adding the identified regulatory factors to the cell culture of adult islet progenitor cells in order to analyze the respective importance of each factor.

Treatment and Diagnosis:

In accordance with the present invention the islet cells, among which are alpha-cells, beta-cells, delta-cells and pp-cells and progenitor cells, among which are alpha-cell progenitor cells, beta-cell progenitor cells, delta-cell progenitor cells and pp-cell progenitor cells find numerous applications in the medical field. An increased amount of beta cells represents an increased potential for insulin secretion, which can be useful in the treatment of e.g. diabetes. In a preferred embodiment, the islet cells or islet progenitor cells are beta-cells.

In one aspect, the invention therefore relates to the use of islet progenitor cells or pancreatic islet cells, such as alpha-cell progenitor cells, beta-cell progenitor cells, delta-cell progenitor cells and pp-cell progenitor cells or alpha-cells, beta-cells, delta-cells and pp-cells, respectively, as claimed herein as a medicament.

In another aspect, the invention relates to the use of progenitor cells or pancreatic islet cells, such as alpha-cell progenitor cells, beta-cell progenitor cells, delta-cell progenitor cells and pp-cell progenitor cells or alpha-cells, beta-cells, delta-cells and pp-cells, respectively, as claimed herein for the manufacture of a medicament for the treatment and/or diagnosis of diseases associated with reduced islet (beta) cell functioning and/or reduced islet (beta) cell mass, such as e.g. diabetes, e.g. diabetes type I and/or type II.

In yet another aspect, the invention provides a pharmaceutical composition for treating or diagnosing diseases associated with reduced islet cell functioning and/or reduced islet cell mass, such as e.g. diabetes, e.g. diabetes type I and/or type II, comprising islet progenitor cells or islet cells, such as alpha-cell progenitor cells, beta-cell progenitor cells, delta-cell progenitor cells and pp-cell progenitor cells or alpha-cells, beta-cells, delta-cells and pp-cells, respectively, respectively, as claimed herein.

The invention also provides a method for treating and/or diagnosing diabetes, e.g. type I and/or type II diabetes, comprising administering to a subject in need thereof an effective amount of
islet progenitor cells, such as alpha-cell progenitor cells, beta-cell progenitor cells, delta-cell progenitor cells and pp-cell progenitor cells, preferably beta-cell progenitor cells,
(pancreatic) islet cells, such as alpha-cells, beta-cells, delta-cells or pp-cells, preferably beta-cells, and/or
a pharmaceutical composition as defined herein.

In yet another aspect the invention provides explanted embryonic pancreas obtained from a mammal, characterized in that said embryonic pancreas comprises injected progenitor cells as claimed herein. The progenitor cells can be provided in the embryonic pancreas by injection. The present explanted embryonic pancreas as claimed herein can be advantageously used for identifying factors that stimulate differentiation and/or proliferation of said progenitor cells.

The invention further provides for a method of treatment of a subject having a reduced islet (e.g. alpha-, beta-, delta- or pp-) cell capacity or (e.g. alpha-, beta-, delta- or pp-) cell mass, with a composition of regulating factors capable of inducing differentiation of progenitor islet (e.g. alpha-, beta-, delta- or pp-) cells in the adult pancreas. Preferably, this treatment is specific to beta cell progenitors, by local administration in e.g. the pancreatic duct or by specific targeting of progenitors, based on their specific plasma membrane determinants. A method of treatment of a patient having a disease associated with reduced islet (e.g. alpha-, beta-, delta- or pp-) cell functioning and/or reduced islet (e.g. alpha-, beta-, delta- or pp-) cell mass, with a composition comprising the factors identified by the methods of the invention.

In a further embodiment, the invention provides the use of the factors identified by the method above in the preparation of a medicament for the treatment of a patient having a disease associated with reduced islet (e.g. alpha-, beta-, delta- or pp-) cell functioning and/or reduced islet (e.g. alpha-, beta-, delta- or pp-) cell mass. Preferably the islet cells are beta-cells.

Explanted Embryonic Pancreas:

In a further embodiment, the explanted embryonic pancreas is used as a microenvironment for (further) differentiation of microinjected stem/progenitor cells, such as embryonic stem cells and bone marrow-derived multipotent progenitor cells, eventually previously partially differentiated, towards islet (e.g. alpha-, beta-, delta- or pp-) cells. Preferably the islet cells are beta-cells.

Upstream Progenitor Cells:

In addition, the invention provides for the identification, isolation, characterization, proliferation and differentiation of stem/progenitor cells that are located upstream in the development of the adult Ngn3 cells using similar tracing technology as described, being guided by the sequence of transcription factors expressed during the embryogenesis. Candidate precursor cells are the Pdx1-positive cells. It has been shown that a Pdx1 knock-out mouse does not develop pancreatic tissue and these cells are therefore likely the precursors of all pancreatic cell types, including the Ngn-3 positive precursors of beta-cells. The advantage of using stem/progenitor cells that are located upstream in the development of the adult Ngn3 cells is that these cells actively divide and are easier to culture in in-vitro systems, whereas the Ngn3-positive adult progenitor cells do not proliferate and can only be cultured efficiently in a pancreatic explant. The model system of the invention provides the necessary tools and information for the successful identification of such progenitor cells, their culturing and differentiation in in-vitro systems towards functional islet (e.g. alpha-, beta-, delta- or pp-) cells. Preferably the islet cells are beta-cells.

The following example is intended to illustrate particular embodiments of the invention, and does not limit the scope of the invention. Those knowledgeable in the treatment and prevention of diseases associated with reduced islet (e.g. alpha-, beta-, delta- or pp-) cell functioning as well as other specialties may find other methods of practicing the invention. However, those methods are deemed to be within the scope of this invention.

### EXAMPLE

### Example 1

### Generation of Beta Cells from Endogenous Progenitors in Adult Mouse Pancreas

Numerous mechanisms that control the differentiation of endocrine progenitor cells in the embryonic pancreas have been disclosed but the knowledge on the existence of post-natal precursors and the generation of islet cells depends merely on descriptive data and indirect proof. Long-term culture of heterogeneous populations of pancreas cells favors enrichment of beta cell-like phenotypes that under certain conditions were able to reverse hyperglycemia when transplanted in diabetic mice. None of these studies, however, was conclusive in demonstrating the existence and origin of a bona fide beta cell progenitor in adult pancreas. The elusiveness of this cell type reached a summit when genetic lineage tracing provided evidence that pre-existing beta cells, rather than stem/progenitor cells are the major source of new beta cells in adult mice both under normal physiological conditions and after 70% pancreatectomy. The transient appearance of a phenotype with non-endocrine or mesenchymal features during the process of beta cell neogenesis in culture further obscured the issue.

Two major problems are at the basis of this complex scenario: the slow turnover of adult beta cells and the lack of specific markers to trace their origin.

Using the surgical technology of partial duct ligation in adult mice, whereby the tail section of the pancreas is surgically isolated from the rest of the pancreas, causing inflammation and forcing regeneration of pancreatic tissue, the applicant has overcome these hurdles. The present example illustrates:
i) adult pancreas comprises endogenous progenitors of pancreatic beta cells, identified after a partial duct ligation procedure
ii) expansion of the beta cell mass following partial duct ligation (PDL) in the pancreas of adult mice, and
iii) the identification of such progenitors using transgenic reporter mice that allow tracing of the promoter activity of Ngn3 as a marker of adult progenitor cell recruitment. Ngn3 is an essential master switch for differentiation of embryonic islet cell progenitors and is extremely rare in normal post-natal pancreas.
iv) the specific isolation and subsequent culturing of said progenitors cells in a pancreatic explant in vitro, whereby the progenitors effectively differentiate into beta cells, producing insulin,
v) the identification of the regulatory factors such as growth factors, cytokines, chemokines etc. that induce this differentiation process.

### MATERIALS AND METHODS

### Mouse Manipulations

The pancreatic duct of 8 weeks old mice (Balb/C, C57BL/6×CD1 Ngn3-LacZ or Ngn3-eGFP) was ligated as described in the art with some minor modifications. Following clamping of the distal bile duct, 60 μl of 5×10⁶ TU of recombinant lentiviruses that express shRNA interfering with Ngn3 (Le-shNgn3) or a scrambled control sequence (Le-scr) were slowly injected in the pancreatic duct. From E12.5 or E13.5 embryos of WT or Ngn3^{-/-} mice, the dorsal lobes of pancreas were isolated as described, cultured in RPMI1640+ 10% fetal calf serum (Hyclone) and micro-injected (Eppendorf TransferMan NK) with 500 GFP/LSSC cells that were collected in a micropipette with 20 μm diameter.

Isolation of Ngn3-eGFP Cells

GFP/LSSC cells were obtained from embryonic (E13.5) and adult (PDL D7) pancreas of Ngn3-eGFP mice following dissociation to single cells (collagenase, 0.3 mg/ml and trypsin, 10 μg/ml, Sigma), filtration (30 μm), incubation with PI (2 μg/ml, Sigma) and TSQ (2 μg/ml, Molecular Probes) for 15 min and sorting on a FACSAria (Becton Dickinson).

### RNA and Protein Analysis

Total RNA was isolated from tissue (RNeasy, Qiagen) or cells (Picopure, Arcturus). Only RNA with RIN number >8 (2100 BioAnalyzer, Agilent) was further analyzed. cDNA synthesis and RT-PCR were done as described in the art using specific primers (see table 1). Quantitative PCR was performed using mouse-specific primers and probes recognizing insulin 1 (Mm01259683), insulin 2 (Mm00731595), glucagon (Mm00801712) and cyclophilin A (Mm02342429) with TaqMan Universal PCR master mix on an ABI Prism 7700 Sequence Detector and data were analyzed using the Sequence Detection Systems Software, Version 1.9.1 (all Applied Biosystems). For analysis of Ngn3: forward primer 5′-GTCGGGAGAACTAGGATGGC-3′ (SEQ ID NO:1), reverse primer 5′-GGAGCAGTCCCTAGGTATG-3′ (SEQ ID NO:2) and probe 5′-CCGGAGCCTCGGACCACGAA-3′ (SEQ ID NO:3). The abundance of Ngn3, insulin 1, insulin 2 and glucagon transcripts was normalized versus the abundance of the transcript encoding the housekeeping protein cyclophilin A.

TABLE 1

primer sequences and amplicon size (bp) in RT-PCT

| | | | |
|---|---|---|---|
| Template | Forward (5′-3′) | reverse (5′-3′) | amplicon |

| | | | |
|---|---|---|---|
| Cyclophilin | TGTCCACAGTCGGAAATGGTGA | ATTCCAGGATTCATGTGCCAG | 317 |
| A | (SEQ ID NO: 4) | (SEQ ID NO: 5) | |
| Neurogenin | CAGTCACCCACTTCTGCTTC | GAGTCGGGAGAACTAGGATG | 159 |
| 3 | (SEQ ID NO: 6) | (SEQ ID NO: 7) | |
| Insulin | CGAGGCTTCTTCTACACACC | GAGGGAGCAGATGCTGGT | 154 |
| | (SEQ ID NO: 8) | (SEQ ID NO: 9) | |
| Glucagon | CCACTCACAGGGCACATTCA | GTCCCTGGTGGCAAGATTGT | 344 |
| | (SEQ ID NO: 10) | (SEQ ID NO: 11) | |
| NeuroD1 | GTCCCAGCCCACTACCAATT | CGGCACCGGAAGAGAAGATT | 440 |
| | (SEQ ID NO: 12) | (SEQ ID NO: 13) | |
| IA1 | CGGGCGCTGCTGCTGTCAC | CCGGCGAGCCCAGGTTGAGG | 301 |
| | (SEQ ID NO: 14) | (SEQ ID NO: 15) | |
| Ptf1a | AACCAGGCCCAGAAGGTTAT | CCTCTGGGGTCCACACTTTA | 150 |
| | (SEQ ID NO: 16) | (SEQ ID NO: 17) | |
| Sox9 | GGCGGAGGAAGTCGGTGAAG | GGGTGCGGTGCTGCTGAT | 449 |
| | (SEQ ID NO: 18) | (SEQ ID NO: 19) | |
| HNF6 | GCAATGGAAGTAATTCAGGGCAG | CATGAAGAAGTTGCTGACAGTGC | 461 |
| | (SEQ ID NO: 20) | (SEQ ID NO: 21) | |
| HB9 | CAAGCTCAACAAGTACCTGTCTCG | GCACCATTGCTGTACGGGAAGTTG | 344 |
| | (SEQ ID NO: 22) | (SEQ ID NO: 23) | |
| Pdx1 | AGGTCACCGCACAATCTTGCT | CTTTCCCGAATGGAACCGA | 375 |
| | (SEQ ID NO: 24) | (SEQ ID NO: 25) | |
| PAX4 | TGAAGTGCCCGAAGTACTCGA | AGGCAGCAGATTGTGCAGCTA | 317 |
| | (SEQ ID NO: 26) | (SEQ ID NO: 27) | |
| Arx | AGTGGCGCAAGCGGGAGAAG | GGCGGGTGTGGGCTGTCT | 376 |
| | (SEQ ID NO: 28) | (SEQ ID NO: 29) | |
| PAX6 | AACAACCTGCCTATGCAACC | ACTTGGACGGGAACTGACAC | 206 |
| | (SEQ ID NO: 30) | (SEQ ID NO: 31) | |
| Nkx2.2 | CTAAATATTTATGGCCATGTACACG | GTTCCAAGCTCCGATGCTCAGGAG | 307 |
| | (SEQ ID NO: 32) | (SEQ ID NO: 33) | |
| Nkx6.1 | CCGGTCGGACGCCCATC | GAGGCTGCCACCGCTCGATTT | 467 |
| | (SEQ ID NO: 34) | (SEQ ID NO: 35) | |
| Amylase | TTCTCCCAAGGAAGCAGACCT | GCCATTCCACTTGCGGATA | 150 |
| | (SEQ ID NO: 36) | (SEQ ID NO: 37) | |
| eGFP | CGTCGCCGTCCAGCTCGACCAG | CATGGTCCTGCTGGAGTTCGTG | 635 |
| | (SEQ ID NO: 38) | (SEQ ID NO: 39) | |
| CAII | GACCCTTGCTCCCTTCTTCC | TGTCCACCATCGCTTCTTCA | 193 |
| | (SEQ ID NO: 40) | (SEQ ID NO: 41) | |

Samples for immunohistochemistry (IHC) were fixed in 4% para-formaldehyde (PFA) for 4 h resp. at RT following embedding in paraffin or at 4° C. followed by ON in 20% sucrose and freezing. Samples for immunocytochemistry (ICC) were fixed in 4% PFA for 10 minutes. Paraffin sections (4 μm) were incubated with antisera specific for insulin (1/5000, guinea pig), glucagon (1/3000, rabbit) and somatostatin (1/5000, rabbit), (generated at the Diabetes Research Center, Brussels), pancreatic polypeptide (1/5000, rabbit, gift from Lilly), synaptophysin (1/50, rabbit, Zymed), pan-keratin (1/1000, rabbit, Dako Cytomation), amylase (1/500, rabbit, Sigma), PHH3 (1/400, rabbit, Upstate Biotechnology), BrdU (1/10, mouse, Cappel), GFP (1/100, rabbit or goat, Abcam) and Ngn3 (1/2000, mouse, Ole Madsen, Hagedorn Research Institute, Gentofte). Antigen retrieval was required for recognition of synaptophysin, PHH3 and Ngn3 (microwave), BrdU and pankeratin (proteinase K). Secondary antibodies for detection of guinea pig, rabbit, goat or mouse antibodies were labeled by fluorescence (Cy3, Cy2 or AMCA) (Jackson ImmunoResearch Labs) or by ABC/DAB (DakoCytomation/Becton Dickinson). Signals of Ngn3 were amplified using the TSA-Cy3 System (Perkin Elmer Life). Images were viewed using normal (Zeiss Axioplan 2) or confocal scanning (Leica DMIRE) microscopy and morphometrically analyzed using NIH ImageJ (vs. 1.3.1). For electron microscopy samples were prepared as known in the art.

Quantitative analysis of the beta cell mass (calculated on the basis of at least 9 sections, 150 μm apart from each other, per pancreas tail or head) and the number of BrdU⁺ insulin³⁰ cells was done as described in the prior art (FIG. 11).

Insulin content of adult and embryonic pancreas and medium insulin were determined by radioimmunoassay using mouse insulin RIA kit (Linco Research Inc).

Glucose response of adult and embryonic GFP/LSSC cells, cultured in Ngn3^{-/-} explants for 1 or 7 days, was assayed for insulin release in the medium following incubation with 6 or 20 mmol/L glucose during the last 24 h. Positive and negative controls were sham-injected embryonic pancreas explants from WT and Ngn3^{-/-} mice, respectively.

### DATA ANALYSIS

All values are depicted as mean±standard error of the mean (s.e.m.) from ≧3 independent experiments and considered significant if p<0.05. All data were statistically analyzed by multi-variate comparison (2-way ANOVA) with Bonferroni correction or 1-way ANOVA with Newman-Keuls correction.

### RESULTS

### Activation of NGN3 Gene Expression Induces Beta Cell Hyperplasia in Adult Mice

Pancreatic beta cells have a slow turnover under normal physiological conditions but expand rapidly under certain experimental conditions like partial duct ligation (PDL). In Balb/c mice, the duct leading to the pancreatic tail was closed while the organ's head located adjacent to the stomach and duodenum remained unaffected. Within one week far most of the acinar exocrine cells underwent apoptosis and likely were scavenged by CD45+ cells recruited to the ligated tail part of pancreas. Moreover, duct cell cycle activity was strongly elevated (FIG. 6E) and consequently, the density of duct structures significantly increased (FIG. 1A). The weight of the pancreatic tail decreased, (FIG. 6A) while body weight and glycemia remained unaffected (FIG. 6B,C). The total insulin+ cell mass in the ligated part of pancreas increased more than 2-fold within one week following surgery (FIG. 1B) and the absolute amount of immunoreactive insulin had doubled one week later, a lag period likely needed for beta cell maturation (FIG. 1C). The individual beta cell size remained similar under all conditions tested (FIG. 6D) meaning that the treatment induced an increase in cell number rather than in cell size. During this period, the number of beta cells in active cell cycle increased 10-fold as established by incorporation of the nucleotide analogue BrdU (FIG. 1D). As PDL robustly induced generation of new beta cells, we investigated the importance of progenitor cell activation and beta cell proliferation in doubling of the beta cell mass. A strong activation of expression of Ngn3, a well-established marker for embryonic islet cell progenitors, was observed specifically in the ligated part of adult mouse pancreas within 3 days following injury. Maximal levels of Ngn3 transcript were reached within one week and subsequently decreased slowly (FIG. 1E). Besides neoformation of beta cells, the number of apoptotic beta cells increased significantly. Apoptotic beta cells were not preferentially in active cell cycle. To investigate the causal relationship between the doubling of beta cell mass and the activation of Ngn3 gene expression, the latter was knocked-down during PDLinduced formation of endocrine pancreas. Recombinant lentiviruses that encode 2 Ngn3-specific short hairpin (sh) interfering RNA molecules (Le-sh1 Ngn3 and Lesh2Ngn3), or a control, scrambled sequence (Le-scr) were injected into the pancreatic duct via the papilla Vateri, followed by ligation of the tail duct. The viruses constitutively express the reporter protein eGFP that allowed us to evaluate the efficiency and specificity of infection in the whole organ and in tissue sections. Injection of reporter virus in sham-treated pancreas transduced 62% of total cells. When virus injection was combined with duct ligation, 18±10% of total cells expressed detectable levels of eGFP one week after surgery (FIG. 2A). Following infection with lentivirus expressing short hairpin RNA, acinar cells disappeared similarly as in control PDL pancreas and no off-target effects on differentiation and proliferation of duct cells were observed. In Le-shNgn3-injected pancreas the Ngn3 transcript abundance was 70±11% (sh1) and 49±4% (sh2) lower than in sham- and Le-scr-injected PDL pancreas at day 7 (FIG. 2B, upper and S5). PDL-induced increase of the beta cell mass was prevented by 66±17% (sh1) and 26±10% (sh2) following infection with Le-shNgn3 (FIG. 2B, middle). These data strongly suggest that beta cell formation following PDL depends at least partly on Ngn3 activity. The induced BrdU labeling index of insulin+ cells decreased with 77±10% (sh1) and 32±12% (sh2) by Le-shNgn3 vs Le-scr injection (FIG. 2B, lower), indicating that an important fraction of BrdU+ beta cells (FIG. 1D) were derived from differentiated Ngn3+ cells.

NGN3+ Cells in Adult Pancreas Originate from Hormone-Progenitors Near Ducts and Become Islet Cells

Given the activated Ngn3 expression in injured pancreas of adult mouse we attempted to track these islet progenitor cells in transgenic Ngn3-nLacZ mice, expressing a nuclear b-galactosidase (bGal) reporter protein under control of a 6.9 kb genomic sequence that includes the Ngn3 promoter and faithfully recapitulates the spatial expression of Ngn3 in the embryonic as well as in the adult pancreas (GM and GG, unpublished data). Histochemistry for bGal activity revealed blue nuclei in adult mouse duodenum (data not shown), known to constitutively express Ngn3 in enteroendocrine progenitor cells. The Ngn3 reporter was also detected in the ligated tail of PDL pancreas but not in the unligated head or in the pancreatic tail of shamoperated mice. The localization of 785 bGal+ cells was examined in 6 mice, 7 days following PDL (FIG. 3A). Of all bGal+ cells 15±1% were immunoreactive for duct cellspecific cytokeratins (CK) (FIG. 3B) and half of the bGal+CK+ cells were lining the duct lumen as shown by confocal scanning microscopy (FIG. 3C). Furthermore, Ngn3 was expressed in duct-lining cells that activated Pdx1 expression following PDL (FIG. 3I). No marker typical for any pancreatic cell type was expressed on 51±2% bGal+ cells (FIG. 3D), one third of which were still in contact with CK+ duct cells (FIG. 3B). Immunohistochemical staining with a Ngn3-specific antibody showed that the Ngn3+ cells in duct-ligated pancreas were devoid of islet cell-specific hormones (FIG. 3E-F). On the other hand, the long half-life of the reporter protein bGal (Gonda et al., 1989) allowed tracing the fate of Ngn3+ cells to their endocrine descendants. Indeed, 34±4% of bGal+ cells contained transcripts encoding islet hormones at day 7 following PDL (FIG. 3G,H). Half the number of hormone expressing bGal+ cells was still in contact with duct cells but none of these were part of the luminal lining. No bGal+ cells co-stained for amylase (not shown). While these lineage tracing data strongly suggest that the observed Ngn3+ cells originate from islet hormone-cells among the lining of ducts and migrate to become hormone+ cells within the islet structures in adult mice they do not fully exclude the alternative possibility that endocrine cells dedifferentiated to Ngn3+ cells. Therefore, we traced permanently labeled beta cells from INS-Cre/R26R mice with PDL and found the label absent from Ngn3+ cells (FIG. 7B) supporting differentiation of Ngn3+-to-islet cells.

NGN3+ Progenitor Cells can be Purified from Adult Mouse Pancreas

Based on the number of bGal+ cells in 30 tissue sections from the PDL pancreas of 3 mice, approximately 5000 bGal+ cells were present in the ligated tail, a sufficiently high number to endeavor their isolation. By flow cytometry, Ngn3+ cells were isolated from the PDL pancreas of reporter mice that express eGFP under control of the same 6.9 kb Ngn3 promoter fragment as used in the Ngn3-nLacZ mice. As for bGal, the half-life of eGFP exceeds that of Ngn3 and consequently the reporter protein was still present in a fraction of the hormone-positive descendants of the pre-endocrine cells (data not shown). These GFP+ cells with hormone-containing vesicles were excluded and only non-granulated GFP+ cells were considered as endocrine progenitors. Seven days following partial duct ligation, PI−/GFP+/TSQ−/LowSSC cells (termed GFP/LSSC cells from hereon) that were viable, green fluorescent and contained only few granules could be isolated from PDL pancreas of Ngn3-eGFP mice (FIG. 4A). The transcript encoding Ngn3 was 200-fold enriched and those encoding insulin and glucagon were very rare in the progenitor population as compared to non-sorted PDL pancreas cells (FIG. 4B). A similar cell population was isolated from the pancreas of Ngn3eYFP/+ knock add-on mice (Mellitzer et al., EMBO J. 25:1344-1352, 2004), corroborating faithful recapitulation of Ngn3 expression in the Ngn3-eGFP mice (FIG. 4D and data not shown). The GFP/LSSC cell population represented 0.04% of the total number of sorted pancreatic cells. Of this sorted population, 93±4% immunostained GFP+, 90.1±5% Ngn3+ and none were insulin+ (FIG. 4E). As expected, the fraction of insulin+ cells was high in the GFP+/TSQ+/HSSC (termed GFP/HSSC from hereon) cell population (85±10%) and in total pancreas (3.0±0.8%). GFP/LSSC cells, sorted from GCG-Cre/R26R/Ngn3-eGFP mice pancreas (FIG. 7C) 7 days following PDL, lacked bGal (800 cells counted) while the reporter was expressed in 5% of islet cells (FIG. 7D). In addition to the differentiation of Ngn3+-to-beta cells (FIG. S6B) and the similar amount of Ngn3 transcripts in islets isolated from the tail of ligated and sham-treated pancreas (lower than in total PDL pancreas) (FIG. 4C), these data provide strong evidence against islet-to-Ngn3+ cell dedifferentiation. Due to the controversial aspect of progenitor cells in adult pancreas, we compared these GFP/LSSC cells with Ngn3+ cells isolated from E13.5 embryonic pancreas and from adult duodenal crypt region, as these Ngn3+ cell types are generally accepted to be genuine endocrine progenitors (Gu et al., Development 129:2447-2457, 2002; Jenny et al., EMBO J. 21:6338-6347, 2002; Schonhoff et al., Dev. Biol. 207:443-454, 2004). 99.8% of the sorted cells from embryonic pancreas and adult duodenum were GFP-positive while none immunostained positive for insulin. The abundance of Ngn3 transcripts in these cells was more than 100-fold higher than in the non-sorted cell populations (data not shown). Electron micrographs of GFP/LSSC cells isolated from embryonic and adult PDL pancreas showed rounded cells that were 3-fold smaller than adult mouse beta cells (985±112 mm3 vs. 3052±178 mm3) and had relatively large nuclei (695±98 mm3 vs. 565±76 mm3) with a remarkable amount of heterochromatin in the periphery of the nuclei (FIG. 4F). The ultra-structural features of Ngn3+ cells isolated from adult duodenum were similar to those of the pancreatic GFP/LSSC cells (data not shown). In contrast to the many dark secretory granules containing mature insulin that are present in all differentiated beta cells, most GFP/LSSC cells from embryonic or adult pancreas were non-granulated and few granules were found in only 5±1% of them. The latter had inclusions of low electron density, without a halo (FIG. 4F), typical for cells with unprocessed hormone (Orci et al., Cell, 42:671-681, 1985) and another indication of the immature cell state. Ngn3 cells isolated from adult regenerating pancreas thus strongly resemble progenitors of endocrine cells in embryonic pancreas and adult duodenum. More extensive gene expression profiling revealed that transcription factors expressed upstream of Ngn3 in early pancreas epithelium (Ptf1a, Sox9, HNF6 and Nkx6.1) were also enriched in GFP/LSSC cells while the ones that continue to be expressed in mature islet cells (Hlxb9 and Pdx1) were higher in GFP/HSSC than LSSC cells. Transcription factors acting downstream of Ngn3 (IA1, Pax4, Arx, Nkx2.2, NeuroD1, Pax6) were overall low in GFP/LSSC cells, also illustrating their early endocrine differentiation status (FIG. 4G).

NGN3+ Cells from Adult Pancreas Differentiate to Functional Islet Cells In Vitro

When cultured in 1 or 10% serum, either in suspension, as monolayer or in 3D collagen gel, over 90% of the GFP/LSSC cells died after one day (data not shown). All factors required for endogenous Ngn3+ cells to survive and differentiate into islet cells should, however, be present in the embryonic pancreas in situ but also in embryonic organ culture (FIG. 8A-C). We therefore considered the ex vivo cultured embryonic mouse pancreas as an appropriate microenvironment to investigate the capacity of the Ngn3+ cells isolated from adult mouse pancreas to differentiate into mature islet cells (FIG. 5A). In wild-type (WT) E12.5 pancreatic explants, the differentiating endocrine cells derived from endogenous Ngn3 expressing cells since no islet hormone+ cells appeared in explants from Ngn3 homozygous null-mutant embryos (FIG. 5B). To exclude interference with these endogenous embryonic Ngn3+ cells, the isolated GFP/LSSC cells from normal embryonic and ligated adult pancreas of Ngn3− eGFP mice were micro-injected in embryonic Ngn3−/− pancreas. No insulin or glucagon peptide or transcript could be detected in the engrafted Ngn3−/− explants following 1 day of culture (FIG. 5B,C). After 7 days of culture, WT explants as well as engrafted—but not sham-injected-Ngn3−/− explants contained transcripts encoding the 4 islet hormones as well as their corresponding peptides (FIG. 5B,C). No cell expressed more than one hormone simultaneously (data not shown). We further examined whether the observed endocrine differentiation was cell autonomous or whether fusion or signaling between injected adult Ngn3+ cells and explanted embryonic pancreas was involved. Firstly, when GFP/LSSC cells were pre-incubated with CellTracker Orange (CMTMR) and injected in explanted pancreas of Ngn3−/− embryonic mice, the injected GFP/LSSC cells differentiated since some of them expressed insulin already at day 4 of culture (FIG. 8). Secondly, when mouse GFP/LSSC cells were cultured in explants of rat embryonic pancreas and differentiating beta cells were immunostained by speciesspecific antibodies directed against insulin C-peptide (Blume et al., Biomed. Biochim. Acta 49:1247-1251, 1990), both mouse and rat cells independently differentiated to C-peptide+ cells. Finally, when GFP/LSSC cells isolated from Ngn3-eGFP mice that constitutively express bGal were cultured in pancreas explants from embryonic Ngn3−/− mice, all differentiated, hormone+ cells were bGal+. Consequently, the endocrine cells originate directly from the injected GFP/LSSC cells, without cell fusion. When explants were labeled with BrdU during the last 16 h of culture, the injected GFP/LSSC cells did not enter the cell cycle after 1 day while 22±6.2% of the newly differentiated insulin+ cells were active in S-phase at day 7 (FIG. 5D). WT explants contained 137±37 ng of insulin following 7 days of culture (vs 1.2±0.8 ng at day 1) and Ngn3−/− explants supplemented with adult GFP/LSSC cells had 35±7 ng insulin (vs 0.2±0.2 at day 1). To evaluate the degree of differentiation of the GFP/LSSC cells, we measured glucose responsiveness of the insulin release. Glucose induced a 1.5-fold increase of insulin secretion from explanted E12.5 pancreas of WT mice at day 7 of culture (FIG. 5E). Embryonic pancreas from Ngn3−/− mice acquired glucose responsiveness when injected with GFP/LSSC cells from adult Ngn3-eGFP mice (PDL D7) since their insulin release increased 2.6-fold when stimulated with 20 mmol/L glucose (FIG. 5E).

1-29. (canceled)

30. Method for preparing pancreatic islet cells starting from progenitor cells comprising the steps of:
a) isolating progenitor cells from the pancreas of an adult mammal, by the steps of:
i) providing transgenic mammals, preferably animals such as rodents, expressing a reporter gene under the control of a suitable progenitor-cell-specific promoter,
ii) detecting the presence of said progenitor cells by detecting the product of said reporter gene, and
iii) isolating the progenitor cells detected in step ii),
b) inducing differentiation of said isolated progenitor cells into differentiated pancreatic islet cells, and
c) proliferating the differentiated pancreatic islet cells obtained in step b).

31. The method of claim 30, wherein steps b) and c) are done in vitro inside an embryonic pancreas explant from a non-human mammal.

32. Method according to claim 30, wherein said pancreatic islet cells comprise alpha-, beta-, delta, or pp-cells and said progenitor cells comprise alpha-, beta-, delta, or pp-cell progenitor cells.

33. Method according to claim 30, wherein said progenitor cells are isolated using a progenitor-cell-specific marker, such as the Neurogenin-3 (Ngn3) marker, specific for beta-cells.

34. Method according to claim 30, wherein said progenitor cells comprise cells, preferably non-endocrine cells, lining the pancreatic duct.

35. Progenitor cells obtainable by carrying out step a) of the method according to claim 30.

36. Progenitor cells according to claim 35, characterized in that said cells express the Ngn3 marker gene.

37. Pancreatic islet cells, preferably pancreatic beta cells, obtainable by carrying out the method according to claim 30.

38. A medicament comprising the progenitor cells according to claim 35.

39. A medicament comprising the pancreatic islet cells according to claim 37.

40. A method for treating and/or diagnosis of diseases associated with reduced islet (e.g. alpha-, beta-, delta-, or pp-) cell functioning and/or reduced islet (e.g. alpha-, beta-, delta-, or pp-) cell mass, comprising administering to the subject a suitable amount of progenitor cells according to claim 35, wherein the disorder is preferably diabetes.

41. A method for treating and/or diagnosis of diseases associated with reduced islet (e.g. alpha-, beta-, delta-, or pp-) cell functioning and/or reduced islet (e.g. alpha-, beta-, delta-, or pp-) cell mass, comprising administering to the subject a suitable amount of pancreatic islet cells according to claim 37, wherein the disorder is preferably diabetes.

42. Pharmaceutical composition for treating or diagnosing diseases associated with reduced islet (e.g. beta-) cell functioning and/or reduced islet (e.g. alpha-, beta-, delta-, or pp-) cell mass, comprising progenitor cells as claimed in claim 35.

43. Pharmaceutical composition for treating or diagnosing diseases associated with reduced islet (e.g. beta-) cell functioning and/or reduced islet (e.g. alpha-, beta-, delta-, or pp-) cell mass, comprising pancreatic islet cells as claimed in claim 37.

44. The composition according to claim 42, wherein said disease is diabetes.

45. An explanted embryonic pancreas obtained from a mammal, characterized in that said embryonic pancreas comprises injected progenitor cells according to claim 35.

46. A method of identifying factors that stimulate differentiation and/or proliferation of pancreatic islet progenitor cells, using the explanted embryonic pancreas according to claim 45.

47. A method for identifying the regulatory factors for the differentiation of islet (e.g. alpha-, beta-, delta-, or pp-) cell progenitors into biologically active islet cells comprising the steps of:
a) isolating progenitor islet cells from the pancreas of an adult mammal, by the steps of:
i) providing transgenic mammals, preferably animals such as rodents, expressing a reporter gene under the control of a suitable promoter,
ii) detecting the presence of said progenitor cells by detecting the product of said reporter gene, and
iii) isolating the progenitor cells detected in step ii),
b) culturing the cells obtained in step a) in the presence or absence of regulatory factors,
c) monitoring differentiation of the progenitor cells into functional beta cells in order to identify those factors needed for differentiation of progenitor islet cells into functional pancreatic islet cells.

48. The method of claim 47, wherein the regulatory factors are chemokines, cytokines, hormones, steroids, survival factors, transmitters, growth factors, proliferation factors and/or differentiation factors.

49. The method of claim 47, wherein the regulatory factors are selected from the group of IGF1/2 or BPs, MCP, ILL IL6 (LIF, CNTF), IL12, IL18, CXCL1, CXCL15, IFNgamma, TNFalpha/beta, GLP1/2, GIP, glucagon, insulin, somatostatin, gastrin, ghrelin, leptin, PYY, NPY, CCK, fractalkine, HB-EGF, betacellulin, amphiregulin, HGF/SF, NGF, midkine1, pleiotrophin, HDGF, VEGF, P1GF, KITL(SCF), G/M-CSF, FGF, HPL, GMFG, Wnt, TCF, APC, TGFbeta, activinA/B, BMP2/5/6/7/8, nodal, CTGF, GDF1/LASS1, contactin, jagged1, HH, netrin, neogenin, osteopontin, osteoglycin, SDF1, TLR ligands and steroids.

50. A method of treatment of a subject having a disease associated with reduced islet (beta) cell functioning and/or reduced islet (e.g. alpha-, beta-, delta-, or pp-) cell mass, with a composition comprising the factors identified by the method of claim 47.

51. A medicament comprising the factors identified by the method of claim 47, for the treatment of a patient having a disease associated with reduced islet (e.g. alpha-, beta-, delta-, or pp-) cell functioning and/or reduced islet (e.g. alpha-, beta-, delta-, or pp-) cell mass.

52. A method of treating a patient having a disease associated with reduced islet (e.g. alpha-, beta-, delta-, or pp-) cell functioning and/or reduced islet (e.g. alpha-, beta-, delta-, or pp-) cell mass by administering a suitable amount of the factors identified by the method of claim 47.

53. A method of differentiating microinjected progenitor cells towards islet (e.g. alpha-, beta-, delta-, or pp-) cells, using an embryonic explanted pancreas as a microenvironment.

54. The method according to claim 53, wherein the progenitor cells are selected from the group of stem cells, embryonic cells, bone-marrow derived multipotent cells, partially differentiated beta cell progenitors and the like.

55. A method for identifying antigenic determinants that are specific for adult islet (e.g. alpha-, beta-, delta-, or pp-) cell progenitors, comprising the step of:
i) analyzing the transcriptome of an adult islet (e.g. alpha-, beta-, delta-, or pp-) cell progenitor cell,
ii) comparing the transcriptome of i) to the transcriptome of a corresponding embryonic islet cell progenitor of the same stage, and
(iii) comparing the transcriptome of i) to the transcriptome of an adult differentiated islet cell, thereby identifying factors or markers that are specific for adult islet cell progenitors.

56. A method of isolating binding molecules (e.g. antibodies) that specifically recognize surface antigens on adult pancreatic islet (e.g. alpha-, beta-, delta-, or pp-) cell progenitor cells, the surface antigens being identified by the method of claim 55, wherein the binding molecules are preferably selected from the group of monoclonal, polyclonal or single chain antibodies, (photo)aptamers, specifically interacting small molecules, specifically interacting proteins and other molecules that specifically bind to one of the antigenic determinants.

57. A method of isolating binding molecules (e.g. single chain antibodies) that specifically recognize surface antigens on adult pancreatic islet (e.g. alpha-, beta-, delta-, or pp-) cell-progenitors, comprising:
a) isolating islet progenitor cells according to the method of claim 30,
b) immunizing an animal, preferably a mouse, a rat, a rabbit or a camelid such as a camel, a llama or a dromedary with said isolated cells,
c) isolate binding molecules from the blood of said animals that bind to the islet progenitor cells with high specificity and affinity, wherein said binding molecules are preferably selected from the group of monoclonal, polyclonal or single chain antibodies, (photo)aptamers, specifically interacting small molecules, specifically interacting proteins and other molecules that specifically bind to one of the antigenic determinants.

58. A method of isolating adult islet (e.g. alpha-, beta-, delta-, or pp-) cell progenitors in a subject, preferably a human, subject, using the binding molecules (e.g. antibodies) obtained in the method of claim 57.

59. The method of claim 57, wherein the binding molecules are selected from the group of monoclonal, polyclonal or single chain antibodies, (photo)aptamers, specifically interacting small molecules, specifically interacting proteins and other molecules that specifically bind to one of the antigenic determinants.

60. The method of claim 30, wherein steps b) and c) are done in an in vitro cell culturing system, by addition of the necessary regulatory factors.
